(19) United States
(12) Patent Application Publication
(10) Pub. No.: US 2010/0249082 A1
(43) Pub. Date: Sep. 30, 2010

(54) MODULATORS OF HYPOXIA INDUCIBLE FACTOR-1 AND RELATED USES

(75) Inventors: Hans-Jurgen Hess, Old Lyme, CT (US); Sajjat Hussoin, Lexington, MA (US); Oliver Schwarz, Konigs Wusterhausen (DE)

Correspondence Address: CLARK & ELBING LLP, 101 FEDERAL STREET, BOSTON, MA, 02110, US

(73) Assignee: Bionaut Pharmaceuticals INC., Stoneham, MA (US)

(21) Appl. No.: 12/524,916

(22) PCT Filed: Jan. 31, 2008
(86) PCT No.: PCT/GB08/00320

§ 371 Date: May 7, 2010

Related U.S. Application Data
(60) Provisional application No. 60/898,640, filed on Jan. 31, 2007.

(30) Foreign Application Priority Data
Jul. 11, 2007 (GB) 0713463.8

(51) Int. Cl. A61K 31/585; C07J 41/00; A61P 35/00; A61P 27/02; A61P 3/10; A61P 9/12; A61P 37/00; A61P 25/24; A61P 25/08

(52) U.S. Cl. 514/175; 540/106

(57) ABSTRACT

The invention features compounds of formula (I): and pharmaceutically acceptable salts and prodrugs thereof, as well methods for modulating the effects of local and systemic hypoxic events using the compounds.

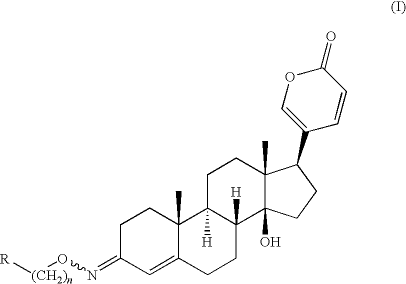

### BACKGROUND OF THE INVENTION

The invention relates to bufadienolide compounds and their use for modulating the effects of local and systemic hypoxic events.

Hypoxia provokes a wide range of physiological and cellular responses in humans and other mammals. The effects of hypoxia vary qualitatively depending on the length of time over which hypoxic conditions are maintained. Acute hypoxia is characterized by increased respiratory ventilation, but after 3-5 minutes, ventilation declines. Individuals exposed to chronic hypoxic conditions undergo a suite of responses including decreased heart rate and increased blood pressure. Metabolically, hypoxia causes decreased glucose oxidation with a shift from oxidative phosphorylation to glycolysis. Glycolysis provides a poorer yield of energy from carbohydrates, and oxidation of fatty acids is greatly reduced. Perhaps for these reasons, hypoxia also triggers increased consumption of carbohydrates. Hypoxia stimulates production of erythropoietin, which in turn leads to an increase in the red blood cell count.

Hypoxia may occur at the level of the whole organism, as, for example, when ventilation is interrupted or when oxygen availability is low. Hypoxia may also occur at a local level essentially any time oxygen consumption outpaces the supply from the bloodstream. Ischemic events are severe forms of local hypoxia that lead to cell death. Recent discoveries relating to the HIF-1 transcription factor have provided considerable insight into the local, cellular response to hypoxia, but our understanding of how the overall physiological response is regulated, and how the systemic and local responses might interact is more limited.

HIF-1 is a transcription factor and is critical to cellular survival in hypoxic conditions, both in cancer and cardiac cells. HIF-1 is composed of the growth factor-regulated subunit HIF-1α, and the constitutively expressed HIF-1β subunit (aryl-hydrocarbon receptor nuclear translocator, ARNT), both of which belong to the basic helix-loop-helix (bHLH)-PAS (PER, ARNT, SIM) protein family. In the human genome, three isoforms of the subunit of the transcription factor HIF have been identified: HIF-1, HIF-2 (also referred to as EPAS-1, MOP2, HLF, and HRF), and HIF-3 (of which HIF-32 also referred to as IPAS, inhibitory PAS domain).

Under normoxic conditions, HIF-1α is targeted for ubiquitinylation by pVHL and is rapidly degraded by the proteasome. This is triggered through post-translational HIF-1α hydroxylation on specific proline residues (proline 402 and 564 in human HIF-1α protein) within the oxygen dependent degradation domain (ODDD), by specific HIF-prolyl hydroxylases (HPH1-3 also referred to as PHD1-3) in the presence of iron, oxygen, and 2-oxoglutarate. The hydroxylated protein is then recognized by pVHL, which functions as an E3 ubiquitin ligase. The interaction between HIF-1α and pVHL is further accelerated by acetylation of lysine residue 532 through an N-acetyltransferase (ARD1). Concurrently, hydroxylation of the asparagine residue 803 within the C-TAD also occurs by an asparaginyl hydroxylase (also referred to as FIH-1), which by its turn does not allow the coactivator p300/CBP to bind to HIF-1 subunit. In hypoxic conditions, HIF-1α remains not hydroxylated and does not interact with pVHL and CBP/p300.

Following hypoxic stabilization, HIF-1α translocates to the nucleus where it heterodimerizes with HIF-1β. The resulting activated HIF-1 drives the transcription of over 60 genes important for adaptation and survival under hypoxia including glycolytic enzymes, glucose transporters Glut-1 and Glut-3, endothelin-1 (ET-1), VEGF (vascular endothelial growth factor), tyrosine hydroxylase, transferrin, and erythropoietin (Brahimi-Horn et al., Trends Cell Biol. 11: S32-S36, 2001; Beasley et al., Cancer Res. 62: 2493-2497, 2002; Fukuda et al., J. Biol. Chem. 277: 38205-38211, 2002; and Maxwell and Ratcliffe, Semin. Cell Dev. Biol. 13: 29-37, 2002).

While HIF-1 is now understood to be the principal mediator of local, or cellular, responses to hypoxia, no global regulator of hypoxia has yet been recognized. It is an object of the invention to identify regulators of hypoxia, and further, to provide uses for such regulators.

Certain compounds are disclosed in Int. Immunopharmac. (2001), 1(1), 119-134 (Terness et al.), Justus Liebigs Annalen der Chemie (1971), 753, 116-34 Goerlich et al.), and in WO 2006/002381-A1 (WARF), WO 2006/120472-A2 (Guy's and St Thomas' NHS Foundation Trust), as well as co-pending applications WO 2007/016656-A2 and WO 2007/081835-A2 (both Bionaut Pharmaceuticals, Inc.). The last relates to modulators of hypoxia inducible factor-1 and related uses, and discloses among other things the HIF-1-modulating compound BP228, shown below:

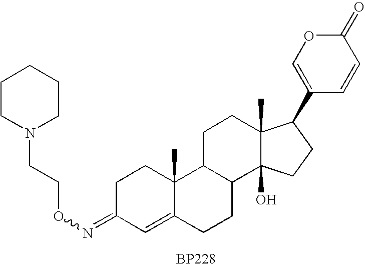

### SUMMARY OF THE INVENTION

The present invention is based on the discovery of further compounds that modulate the effects of local and systemic hypoxic events. Dysregulation (e.g. excessive or insufficient signaling) of the HIF-steroid signaling pathway can contribute, in a downstream fashion, to a wide variety of disorders including, without limitation, cancer, macular degeneration, hyperglycemia, metabolic syndrome (e.g. Syndrome X), cataracts, hypertension, autoimmune disorders, anxiety, depression, insomnia, chronic fatigue, epilepsy, and symptoms associated with irregular angiogenesis. The compounds of the invention, which are modulators (e.g. agonists and antagonists) of the HIF-steroid signaling pathway, can be used to treat these disorders.

Accordingly, in a first aspect the invention features a compound of formula I:

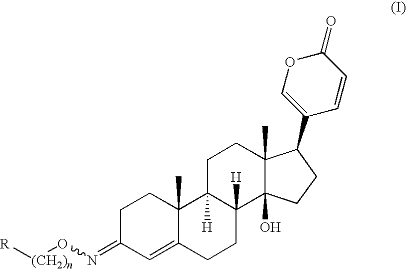

or a pharmaceutically acceptable salt or prodrug thereof, wherein:

R is a nitrogen-containing C₂₋₆ heterocyclyl group joined to the (CH₂)ₙ linkage by means of its nitrogen atom and optionally substituted with a C₁₋₅ alkyl, C₁₋₅ hydroxyalkyl, C₁₋₅ halogenoalkyl or C₁₋₅ acyl group, a phenyl group or a C₁₋₅ alkyl group further substituted with a phenyl group, or

R is a group R′R″N— with R′ and R″ independently equal to H, C₁₋₅ alkyl or methylenecyclopropyl, and

n is an integer in the range 2 to 5,

with the proviso that, when R represents an unsubstituted piperidino group or a dimethylamino group, then n does not equal 2.

Preferably R is a group R¹R²N— with R¹ and R² independently equal to H, C₁₋₅ alkyl or methylenecyclopropyl, or R¹ and R² together with the nitrogen atom connecting them equal to:

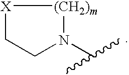

wherein X is CH₂, CH.CH₃, CH.Ph, CH.CH₂.Ph, O or NR³ where R³ is H, C₁₋₅ alkyl, C₁₋₅ hydroxyalkyl, C₁₋₅ halogenoalkyl or C₁₋₅ acyl, and m is 1, 2 or 3.

Preferably R is pyrrolidino, piperidino, morpholino, piperazino or homo-piperazino, optionally substituted with a C₁₋₅ alkyl, C₁₋₅ hydroxyalkyl, C₁₋₅ halogenoalkyl or C₁₋₅ acyl group, a phenyl group or a C₁₋₅ alkyl group further substituted with a phenyl group.

Preferably the compound is of formula Ia:

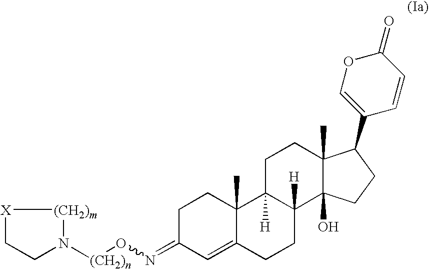

or is a pharmaceutically acceptable salt or prodrug thereof, wherein X is CH₂, CH.CH₃, CH.Ph, CH.CH₂.Ph, O or NR³ where R³ is H, C₁₋₅ alkyl, C₁₋₅ hydroxyalkyl, C₁₋₅ halogenoalkyl or C₁₋₅ acyl, n is an integer in the range 2 to 5 and m is 1, 2 or 3.

Preferably R³ is H or a group the (CH₂)ₚZ where Z is H, OH or halogeno and p is an integer in the range 2 to 5. Preferably p is 2.

Preferably m is 2 and X is CH₂, CH.CH₂.Ph, or O, especially CH₂.

Preferably n is an integer in the range 3 to 5, especially 4.

In another aspect, the invention features a method for treating a disorder in a mammal mediated by hypoxia inducible factor-1 (HIF-1) by administering to the mammal a compound of the invention in an amount sufficient to treat the disorder, and the use of the compound in the manufacture of a medicament for such a method. The disorder can be a metabolic disorder, such as syndrome X, obesity, or atherogenic dyslipidemia. The disorder can be a hypertension disorder, such as sleep-disordered breathing, or obstructive sleep apnea. The disorder can be an inflammatory disorder, such as arthritis, psoriasis, or atherosclerosis. The disorder can be characterized by pathogenic angiogenesis. Disorders characterized by pathogenic angiogenesis include, without limitation, ocular disorders, such as optic disc neovascularization, iris neovascularization, retinal neovascularization, choroidal neovascularization, corneal neovascularization, vitreal neovascularization, glaucoma, pannus, pterygium, macular edema, diabetic macular edema, vascular retinopathy, retinal degeneration, uveitis, inflammatory diseases of the retina, excessive angiogenesis following cataract surgery, and proliferative vitreoretinopathy; and neoplastic disorders, such as carcinoma of the bladder, breast, colon, kidney, liver, lung, head and neck, gall-bladder, ovary, pancreas, stomach, cervix, thyroid, prostate, or skin; a hematopoietic cancer of lymphoid lineage, a hematopoietic cancer of myeloid lineage, a cancer of mesenchymal origin, a cancer of the central or peripheral nervous system, melanoma, seminoma, teratocarcinoma, osteosarcoma, thyroid follicular cancer, and Kaposi's sarcoma. The disorder can be Alzheimer's Disease.

In a related aspect, the invention features a method for reducing VEGF expression in a cell by contacting the cell with a compound of the invention in an amount sufficient to reduce VEGF expression.

In yet another aspect, the invention features a method for treating a patient with a neoplastic disorder by administering to the patient (i) a compound of the invention, and (ii) an antiproliferative agent, wherein the compound of the invention and the antiproliferative agent are administered simultaneously, or within 14 days of each other, each in an amount that together is sufficient to treat a neoplastic disorder. The antiproliferative agent can be selected from alkylating agents, folic acid antagonists, pyrimidine antagonists, purine antagonists, antimitotic agents, DNA topoisomerase II inhibitors, DNA topoisomerase I inhibitors, taxanes, DNA intercalators, aromatase inhibitors, 5-alpha-reductase inhibitors, estrogen inhibitors, androgen inhibitors, gonadotropin releasing hormone agonists, retinoic acid derivatives, and hypoxia selective cytotoxins. Desirably, the antiproliferative agent is gemcitabine.

In another aspect, the invention features a kit including: (i) a compound of the invention; and (ii) instructions for administering the compound of the invention to a patient diagnosed with a disorder mediated by hypoxia inducible factor-1 (HIF-1). The kit can further include an antiproliferative agent, formulated separately or together. Desirably, the compound of the invention and antiproliferative agent are formulated together for simultaneous administration.

In a related aspect, the invention features a method for synthesizing a compound of the invention. The method includes the step of condensing H₂NO(CH₂)ₙR with the corresponding 3-oxo bufadienolide compound.

In the generic descriptions of compounds of this invention, the number of atoms of a particular type in a substituent group is generally given as a range, e.g. an alkyl group containing from 1 to 5 carbon atoms or C₁₋₅ alkyl. Reference to such a range is intended to include specific references to groups having each of the integer number of atoms within the specified range. For example, an alkyl group from 1 to 5 carbon atoms includes each of C₁, C₂, C₃, C₄ and C₅. A C₁₋₅ halogenoalkyl, for example, includes from 1 to 5 carbon atoms in addition a halogeno substituent. Other numbers of atoms and other types of atoms may be indicated in a similar manner.

As used herein, the terms “alkyl” and the prefix “alk-” are inclusive of both straight chain and branched chain groups and of cyclic groups, i.e. cycloalkyl. Cyclic groups can be monocyclic or polycyclic and preferably have from 3 to 5 ring carbon atoms. Exemplary cyclic groups include cyclopropyl, cyclobutyl, cyclopentyl, and cyclohexyl groups. The C₁₋₅ alkyl group may be substituted or unsubstituted. C₁₋₅ alkyls include, without limitation, methyl; ethyl; n-propyl; isopropyl; cyclopropyl; cyclopropylmethyl; cyclopropylethyl; n-butyl; isobutyl; sec-butyl; tert-butyl; cyclo-butyl; cyclobutylmethyl; cyclobutylethyl; n-pentyl; cyclopentyl; 1-methylbutyl; 2-methylbutyl; 3-methylbutyl; 2,2-dimethylpropyl; 1-ethylpropyl; 1,1-dimethylpropyl; and 1,2-dimethylpropyl.

By “C₂₋₆ heterocyclyl” is meant a stable 5- to 7-membered monocyclic or 7- to 14-membered bicyclic heterocyclic ring which is saturated, partially unsaturated or unsaturated (aromatic), and which consists of 2 to 6 carbon atoms and 1, 2, 3 or 4 heteroatoms independently selected from the group consisting of N, O, and S and including any bicyclic group in which any of the above-defined heterocyclic rings is fused to a benzene ring. The heterocyclyl group may be substituted or unsubstituted. The nitrogen and sulfur heteroatoms may optionally be oxidized. The heterocyclic ring may be covalently attached via any heteroatom or carbon atom which results in a stable structure, e.g. an imidazolinyl ring may be linked at either of the ring-carbon atom Positions or at the nitrogen atom. A nitrogen atom in the heterocycle may optionally be quaternized. Preferably when the total number of S and O atoms in the heterocycle exceeds 1, then these heteroatoms are not adjacent to one another. Heterocycles include, without limitation, morpholinyl, 2-pyrrolidonyl, 2H,6H-1,5,2-dithiazinyl, 2H-pyrrolyl, 4-piperidonyl, 6H-1,2,5-thiadiazinyl, homopiperazinyl, imidazolidinyl, imidazolinyl, imidazolyl, 1H-indazolyl, isothiazolyl, isoxazolyl, oxa-diazolyl, 1,2,3-oxadiazolyl, 1,2,4-oxadiazolyl, 1,2,5-oxadiazolyl, 1,3,4-oxadiazolyl, oxazolidinyl, oxazolyl, piperazinyl, piperidinyl, pteridinyl, piperidonyl, 4-piperidonyl, pyrazinyl, pyrazolidinyl, pyrazolinyl, pyrazolyl, pyridazinyl, pyridinyl, pyrimidinyl, pyrrolidinyl, pyrrolinyl, pyrrolyl, 6H-1,2,5-thiadiazinyl, 1,2,3-thiadiazolyl, 1,2,4-thiadiazolyl, 1,2,5-thiadiazolyl, 1,3,4-thiadiazolyl, thiazolyl, triazinyl, 1,2,3-triazolyl, 1,2,4-triazolyl, 1,2,5-triazolyl, 1,3,4-triazolyl.

By “acyl” is meant a chemical moiety with the formula R—C(O)—, wherein R is C₁₋₄ alkyl.

By “halogeno” is meant bromo, chloro, iodo or fluoro.

By “hydroxyalkyl” is meant a chemical moiety with the formula —(R)—OH, wherein R is C₁₋₅ alkylene.

Asymmetric or chiral centers may exist in any of the compounds of the present invention. The present invention contemplates the various stereoisomers and mixtures thereof. Individual stereoisomers of compounds of the present invention are prepared synthetically from commercially available starting materials which contain asymmetric or chiral centers or by preparation of mixtures of enantiomeric compounds followed by resolution well-known to those of ordinary skill in the art. These methods of resolution are exemplified by (1) attachment of a racemic mixture' of enantiomers, designated (±), to a chiral auxiliary, separation of the resulting diastereomers by recrystallization or chromatography and liberation of the optically pure product from the auxiliary or (2) direct separation of the mixture of optical enantiomers on chiral chromatographic columns. Enantiomers are designated herein by the symbols R or S depending on the configuration of substituents around the chiral carbon atom. Alternatively, enantiomers are designated as (+) or (−) depending on whether a solution of the enantiomer rotates the plane of polarized light clockwise or counterclockwise, respectively.

Geometric isomers may also exist in the compounds of the present invention. The present invention contemplates the various geometric isomers and mixtures thereof resulting from the arrangement of substituents around a carbon-carbon double bond and designates such isomers as of the Z or E configuration, where the term Z represents substituents on the same side of the carbon-carbon double bond and the term E represents substituents on opposite sides of the carbon-carbon double bond. In the case of a compound of formula I, the respective forms would generally be:

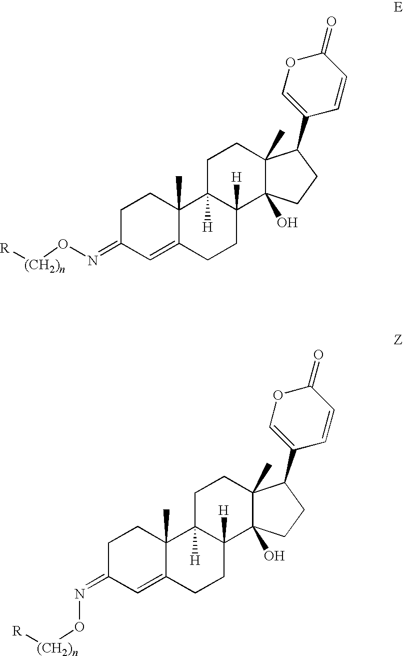

The E and Z isomers appear to interconvert under normal experimental conditions without decomposing, so isolation of individual geometric isomers has not been possible.

It is also recognized that for structures in which tautomeric forms are possible, the description of one tautomeric form is equivalent to the description of both, unless otherwise specified.

As used herein, the term “pharmaceutically acceptable salt” refers to those salts which are suitable for use in contact with the tissues of humans and animals without undue toxicity, irritation, or allergic response. Pharmaceutically acceptable salts are well known in the art. For example, S. M Berge et al. describe Pharmaceutically acceptable salts in detail in J. Pharmaceutical Sciences 66: 1-19,1977. The salts can be prepared in situ during the final isolation and purification of any compound described herein or separately by reacting the free base group with a suitable organic acid.

The term “prodrug,” as used herein, represents compounds which are rapidly transformed in vivo to the parent compound of the above formula, for example, by hydrolysis in blood. Prodrugs of any of the compounds described herein may be conventional esters that are hydrolyzed to their active carboxylic acid form. Some common esters which have been utilized as prodrugs are phenyl esters, aliphatic (C₈-C₂₄) esters, acyloxymethyl esters, carbamates and amino acid esters. In another example, any compound described herein that contains an OH group may be acylated at this Position in its prodrug form. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and Judkins et al., Synthetic Communications 26(23): 4351-4367, 1996, each of which is incorporated herein by reference.

By an amount “sufficient” is meant the amount of a compound of the invention required to treat a disorder mediated by a local or general hypoxic response. This amount, an amount sufficient, can be routinely determined by one of skill in the art, by animal testing and/or clinical testing, and will vary, depending on several factors, such as the particular disorder to be treated and the particular compound of the invention used. This amount can further depend upon the subject's weight, sex, age and medical history.

As used herein, the term “treatment” refers to the administration of a compound of the invention in an amount sufficient to, alleviate, ameliorate, or delay the progress of one or more symptoms or conditions associated with a disorder mediated by a local or general hypoxic response.

The term “administration” or “administering” refers to a method of giving a dosage of a pharmaceutical composition to a subject, where the method is, e.g., topical, transdermal, oral, intravenous, intraperitoneal, intracerebroventricular, intrathecal, or intramuscular. The preferred method of administration can vary depending on various factors, e.g. the components of the pharmaceutical composition, site of administration, and severity of the symptoms being treated.

The compounds of the invention can be more efficacious and more easily administered (e.g. orally) in comparison to the prior art compounds BNC1 and BNC4.

Other features and advantages of the invention will be apparent from the following detailed description, the drawings, and the claims.

### BRIEF DESCRIPTION OF THE DRAWINGS

FIG. 1 is a schematic diagram showing the adaptation of a cell to hypoxia, which leads to activation of multiple survival factors. The HIF family acts as a master switch transcriptionally activating many genes and enabling factors necessary for glycolytic energy metabolism, angiogenesis, cell survival and proliferation, and erythropoiesis. The level of HIF proteins present in the cell is regulated by the rate of their synthesis in response to factors such as hypoxia, growth factors, androgens and others. Degradation of HIF depends in part on levels of reactive oxygen species (ROS) in the cell. ROS leads to ubiquitylation and degradation of HIF.

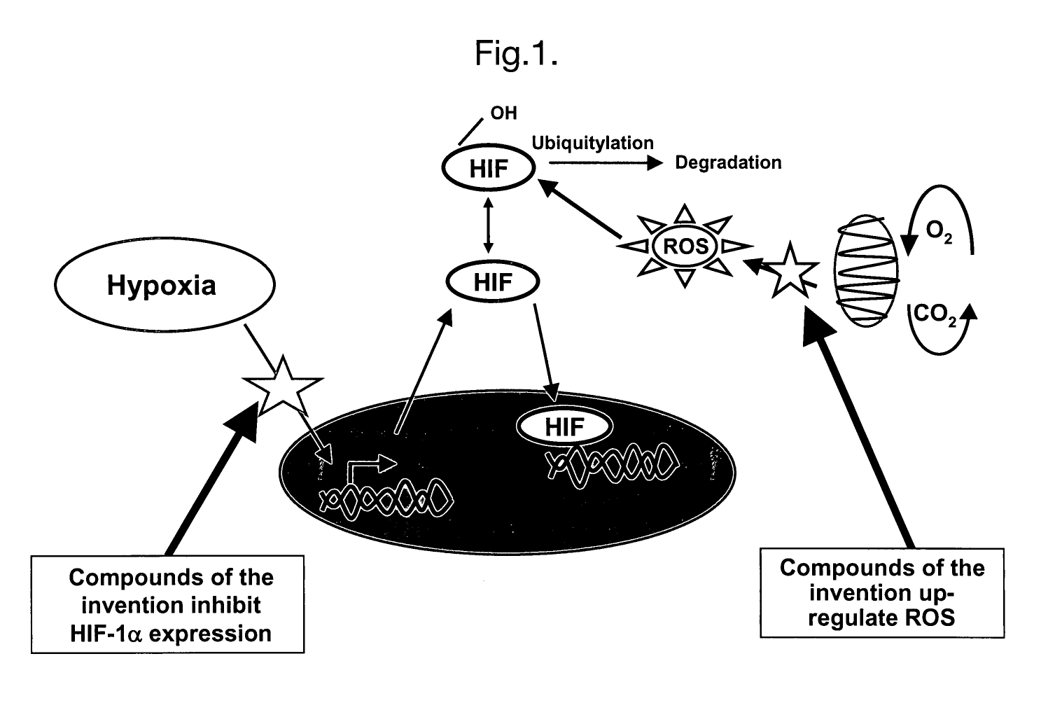

FIG. 2 is a Western blot analysis comparison of ouabain (BNC1) and BNC4 in inhibiting hypoxia-mediated HIF-la induction in human tumor cells (Caki-1 and Panc-1 cells).

FIG. 3 is a Western blot analysis showing that proscillaridin (BNC4) blocks HIF-1α induction by a prolyl-hydroxylase inhibitor (mimosine) under normoxia.

### DETAILED DESCRIPTION

The present invention is based in part on the discovery of compounds which can modulate the effects that are observed as a result of cellular or systemic hypoxia. One salient feature of the present invention is the discovery that certain agents induce an hypoxic stress response and expression of angiogenic factors (such as VEGF) in cells, and that the compounds of the invention can be used to reduce that response. Since hypoxic stress response is associated with the expression of certain angiogenesis factors, including (but not limited to) VEGF, administration of a compound of the invention for inhibiting hypoxic stress response would also inhibit VEGF (and other angiogenesis factors) mediated angiogenesis.

Metabolic Disorders

The compounds of the invention can be useful for the treatment of metabolic disorders such as, for example, hyperglycemia, impaired glucose tolerance, metabolic syndrome (e.g. Syndrome X), glucosuria, metabolic acidosis, cataracts, diabetic neuropathy and nephropathy, obesity, hyperlipidemia, and metabolic acidosis.

Metabolic syndrome X is a constellation of metabolic disorders that all result from the primary disorder of insulin resistance. All the metabolic abnormalities associated with syndrome X can lead to cardiovascular disorders. When present as a group, the risk for cardiovascular disease and premature death are very high. The characteristic disorders present in metabolic syndrome X include: insulin resistance, hypertension, abnormalities of blood clotting, low HDL and high LDL cholesterol levels, and high triglyceride levels. For the treatment of Syndrome X, the compounds of the invention can be used alone, or in combination with any existing anti-diabetic agent. Agents which may be used in combination with the compounds of the invention include, without limitation, insulin, insulin analogs (e.g. mecasermin), insulin secretagogues (e.g. nateglinide), biguamides (e.g. metformin), sulfonylureas (e.g. chlorpropamide, glipizide, or glyburide), insulin sensitizing agents (e.g. PPARγ agonists, such as troglitazone, pioglitazone, or rosiglitazone), α-glucosidase inhibitors (e.g. acarbose, voglibose, or miglitol), aldose reductase inhibitors (e.g. zopolrestat) , metiglinides (e.g. repaglinide), glycogen phosphorylase inhibitors, and GLP-1 and functional mimetics thereof (e.g. exendin-4), among others.

Obesity may result from or be associated with a variety of phenotypes, many of which are reflective of a hypoxic condition. For example, many individuals suffering from chronic hypoxia crave carbohydrates, and carbohydrate cravings are also common in obese individuals. It is thought that adipose tissue exhibits angiogenic activity and also that adipose tissue mass can be regulated via the vasculature. There is reciprocal paracrine regulation of adipogenesis and angiogenesis. Furthermore, it has been shown that a blockade of vascular endothelial growth factor (VEGF) signaling can inhibit in vivo adipose tissue formation. Fukumura et al. in Circulation Research 93: e88-97, 2003.

The present invention features methods for down-regulating angiogenetic factors to inhibit angiogenesis in vivo in treating/preventing obesity, by administering a compound of the invention, with or without other anti-angiogenesis factors.

For the treatment of obesity, a compound of the invention may be used alone, or in combination with any existing anti-obesity agent, such as those described by Flint et al., J. Clin. Invest. 101: 515-520, 1998 or by Toft-Nielsen et al., Diabetes Care 22: 1137-1143, 1999. Agents which may be used in combination with the compounds of the present invention include, without limitation, fatty acid uptake inhibitors (e.g. orlistat), monoamine reuptake inhibitors (e.g. sibutramine), anorectic agents (e.g. dexfenfluramine or bromocryptine), sympathomimetics (e.g. phentermine, phendimetrazine, or mazindol), and thyromimetic agents, among others.

Hypertensive Disorders

The compounds and methods of the invention can be useful for the treatment of hypertension. Systemic hypertension is the most prevalent cardiovascular disorder in the United States, affecting more than 50 million individuals. Hypertension is a common cause of major medical illnesses, including stroke, heart disease, and renal failure, in middle-aged males. Its prevalence in the United States is around 20%, with the rate of newly diagnosed hypertensive patients being about 3% per year.

Obstructive sleep apnea syndrome is common in the same population. It is estimated that up to 2% of women and 4% of men in the working population meet criteria for sleep apnea syndrome. The prevalence may be much higher in older, non-working men. Many of the factors predisposing to hypertension in middle age, such as obesity, are also associated with sleep apnea. Recent publications describe a 30% prevalence of occult sleep apnea among middle-aged males with hypertension. In addition, an association has also been found for hypertension and sleep-disordered-breathing (see, for example, Fletcher, Am. J. Med. 98(2): 118-28, 1995).

HIF-1, as one of the pivotal mediators in the response to hypoxia, has been implicated in the pathogenesis of hypertension (see, for example, Li and Dai, Chin. Med. J. (Engl). 117(7): 1023-8, 2004; and Semenza, Genes and Development 14: 1983-1991, 2000). Due to their ability to decrease HIF-expression, a compound of the invention can be useful for the treatment of disorders caused by hypertension, such as sleep-disordered breathing and obstructive sleep apnea.

Angiogenic Disorders

The compounds of the invention are potent inhibitors of HIF-1, which is itself a potent activator of pro-angiogenic factors. While not wishing to be bound to any particular mechanism, it is reasonable to expect that a factor involved in mounting a global response to hypoxia would suppress local responses, such as angiogenesis, that would be inappropriate if local cellular hypoxia is attributable to systemic disturbances in ventilation or oxygen supply.

The compositions and methods of the invention can be used to inhibit angiogenesis which is nonpathogenic, i.e. angiogenesis which results from normal biological processes in the subject. Besides during embryogenesis, angiogenesis is also activated in the female reproductive system during the development of follicles, corpus luteum formation and embryo implantation. During these processes, angiogenesis is mediated mainly by VEGF. Uncontrolled angiogenesis may underlie various female reproductive disorders, such as prolonged menstrual bleeding or infertility, and excessive endothelial cell proliferation has been observed in the endometrium of women with endometriosis. Neovascularization also plays a critical role in successful wound healing that is probably regulated by IL-8 and the growth factors FGF-2 and VEGF. Macrophages, known cellular components of the accompanying inflammatory response, may contribute to the healing process by releasing these angiogenic factors. Examples of non-pathogenic angiogenesis include endometrial neovascularization, and processes involved in the production of fatty tissues or cholesterol. Thus, the invention provides a method for inhibiting non-pathogenic angiogenesis, e.g. for controlling weight or promoting fat loss, for reducing cholesterol levels, or as an abortifacient.

The compositions and methods of the invention can also be used to inhibit angiogenesis which is pathogenic, i.e. a disease in which pathogenicity is associated with inappropriate or uncontrolled angiogenesis. For example, most cancerous solid tumors generate an adequate blood supply for themselves by inducing angiogenesis in and around the tumor site. This tumor-induced angiogenesis is often required for tumor growth, and also allows metastatic cells to enter the bloodstream. Furthermore, numerous ocular diseases are associated with uncontrolled or excessive angiogenesis.

Neoplastic disorders associated with angiogenesis that can be treated using the compounds and methods of the invention include, without limitation, tumor growth, hemangioma, meningioma, solid tumors, leukemia, neovascular glaucoma, angiofibroma, pyogenic granuloma, scleroderma, trachoma, and metastasis thereof.

Non-neoplastic disorders associated with angiogenesis that can be treated using the compounds and methods of the invention include, without limitation, retinal neovascularization, diabetic retinopathy, retinopathy of prematurity (ROP), endometriosis, macular degeneration, age-related macular degeneration (ARMD), psoriasis, arthritis, rheumatoid arthritis (RA), atherosclerosis, hemangioma, Kaposi's sarcoma, thyroid hyperplasia, Grave's disease, arterioyenous malformations (AVM), vascular restenosis, dermatitis, hemophilic joints, hypertrophic scars, synovitis, vascular adhesions, and other inflammatory diseases.

The compounds and methods of the invention can also be useful for preventing or alleviating abnormal angiogenesis following cataract surgery. In normal lenses, immunoreactivity against bufalin and ouabain-like factor is sevenfold to 30-fold higher in the capsular epithelial layer than in the lens fiber region (Lichtstein et al., Involvement of Na⁺, K⁺-ATPase inhibitors in cataract formation, in Na/K-ATPase and Related ATPases, 2000, Taniguchi, K. & Haya, S., eds, Elsevier Science, Amsterdam). In human cataractous lenses, the concentration of the sodium pump inhibitor was much higher than in normal lenses. Hence, it was isolated from cataractous lenses and identified as 19-norbufalin and its Thr-Gly-Ala tripeptide derivative (Lichtstein et al., Eur. J. Biochem. 216: 261-268, 1993). Cataract surgery will remove such steroids, resulting in the possible loss of the local inhibition of unwanted angiogenesis in the eye. Patients after cataract surgery may therefore be more vulnerable to conditions associated with abnormal angiogenesis.

Inflammatory Disorders

Angiogenesis and enhanced microvascular permeability are hallmarks of a large number of inflammatory diseases. Angiogenesis and chronic inflammation are closely linked (Jackson et al., FASEB J. 11: 457-465, 1997). Angiogenic blood vessels at the site of inflammation are enlarged and hyperpermeable to maintain the blood flow and to meet the increased metabolic demands of the tissue (Jackson et al., Supra). Several proangiogenic factors, including vascular endothelial growth factor (VEGF) (Detmar, J. Dermatol. Sci. 24(suppl 1): S78-S84, 2000; Brown et al., J. Invest. Dermatol. 104: 744-749, 1995; Fava et al., J. Exp. Med. 180: 341-346, 1994) and members of the CXC-chemokine family (Schroder and Mochizuki, Biol. Chem. 380: 889-896, 1999; Strider et al., Shock 4: 155-160, 1995) have been found to be up-regulated during inflammation. While not wishing to be bound by any particular theory, inflammation may induce local hypoxia response and promote angiogenesis through, for example, VEGF and other factors. Furthermore, immune cells tend to have a constitutively high level of HIF-1. This is coupled with a tendency of these cells to rely on glycolysis. Thus, a number of phenomena more typically associated with hypoxic cells are constitutively present in certain immune cells.

Accordingly, the compounds and methods of the invention can be used for the treatment of inflammatory diseases, such as rheumatoid arthritis, psoriasis, and atherosclerosis.

Alzheimer's Disease (AD)

The compounds and methods of the invention can be useful for inhibiting the onset and/or development of AD. Alzheimer's disease (AD), characterized by impairments in cognition and memory, is clearly associated with the slow accumulation of amyloid β peptides (AβPs) in the central nervous system (Selkoe, Physiol. Rev. 81: 741-766, 2001; Small et al., Nat. Rev. Neurosci. 2: 595-598, 2001). AβPs are generated via amyloidogenic processing of amyloid precursor protein (APP) by β- and γ-secretases, and recent evidence suggests that y-secretase activity requires the formation of a complex between presenilin, nicastrin, APH-1 and pen-2 (Edbauer et al., Nat. Cell Biol. 5: 486-488, 2003). Disruption of Ca²⁺ homeostasis has been strongly implicated in the neurodegeneration of AD; indeed, increased Ca²⁺-dependent protease activity occurs in association with degenerating neurones in AD brain tissue (Nixon et al., Ann. N Y Acad. Sci. 747: 77-91, 1994), and AβPs perturb Ca²⁺ homeostasis, rendering cells susceptible to excitotoxic damage (Mattson et al., J. Neurosci. 12: 376-389, 1992). Presenilin mutations are known to have effects on cellular Ca²⁺ homeostasis (Mattson et al., Trends Neurosci. 23,222-229, 2000), and familial AD (FAD)-related mutations of presenilin-1 (PS-1) can alter inositol triphosphate-coupled intracellular Ca²⁺ stores as well as Ca²⁺ influx pathways (Leissring et al., J. Cell Biol. 149: 793-798, 2000; Mattson et al., Trends Neurosci. 23: 222-229, 2000; Yoo et al., Neuron 27: 561-572, 2000): This may contribute to neurodegeneration, since disruption of Ca²⁺ homeostasis is an important mechanism underlying such loss of neurones (Chan et al., J. Biol. Chem. 275: 18195-18200, 2000; Mattson et al., J. Neurosci. 20: 1358-1364, 2000; Yoo et al., supra).

Periods of cerebral hypoxia or ischemia can increase the incidence of AD (Tatemichi et al., Neurology 44: 1885-1891, 1994; Kokmen et al., Neurology 46: 154-159, 1996), and APP expression is elevated following mild and severe brain ischemia (Kogure and Kato, Stroke 24: 2121-2127, 1993). Since the non-amyloidogenic cleavage product of APP (sAPPα) is neuroprotective (Mattson, Physiol. Rev. 77: 1081-1132, 1997; Selkoe, Physiol. Rev. 81: 741-766, 2001), increased expression during hypoxia could be considered a protective mechanism against ischemia. However, increased APP levels would also provide an increased substrate for AβP formation. It was previously shown that AβP formation is increased following hypoxia in PC12 cells (Taylor et al., J. Biol. Chem. 274: 31217-31222, 1999; Green et al., J. Physiol. 541: 1013-1023, 2002). Furthermore, prolonged hypoxia potentiates bradykinin (BK)-induced Ca²⁺ release from intracellular stores in rat type I cortical astrocytes. This was due to dysfunction of mitochondria and plasmalemmal Na⁺/Ca²⁺ exchanger (NCX; Smith et al., J. Biol. Chem. 278: 4875-4881, 2003). Peers et al., Biol. Chem. 385(3-4): 285-9, 2004 report that sustained central hypoxia predisposes individuals to dementias such as Alzheimer's disease, in which cells are destroyed in part by disruption of Ca²⁺ homeostasis. Moreover, hypoxia increases the levels of presenilin-1, a major component of a key enzyme involved in Alzheimer's disease. Thus there is established link between periods of hypoxia and the development of AD.

Proliferative Disorders

The compounds and methods of the invention can be useful for the treatment of proliferative disorders. Notably, the compounds of the invention can inhibit the proliferation of cancer cell lines at a concentration well below the known toxicity level.

Combination Therapy

The compounds of the invention can be used in combination with other antiproliferative agents for the treatment of cancer and/or inhibiting the formation of metastases. Antiproliferative agents to be used in the combination include, without limitation, those agents provided in Table 1.

Desirably, the compound of the invention is added to an existing clinical regimen (e.g. paclitaxel for the treatment of breast cancer) for the purpose of reducing the minimum efficacious dose. The benefit to the patient is an increase in the therapeutic index of the anticancer agent when used in combination with a compound of the invention. Accordingly, the compound of the invention can be added to any existing cancer therapy regimen for the purpose of reducing adverse drug reactions, extending the life of the patient, and/or improving the cure rate.

TABLE 1
Antiproliferative Agents

| Class | Type of Agent | Nonproprietary Names | Cancers |
|---|---|---|---|
| Alkylating | Nitrogen mustards | Mechlorethamine | Hodgkin's disease, non-Hodgkin's |
| agents | | | lymphomas |
| | | Cyclophosphamide, | Acute and chronic lymphocytic, leukemias, |
| | | Ifosfamide | Hodgkin's disease, non-Hodgkin's |
| | | | lymphomas, multiple myeloma, |
| | | | neuroblastoma, breast, ovary, lung, |
| | | | Wilms' tumor, cervix, testis, soft-tissue |
| | | | sarcomas |
| | | Melphalan | Multiple myeloma, breast, ovary |
| | | Chlorambucil | Chronic lymphocytic leukemia, Primary |
| | | | macroglobulinemia, Hodgkin's disease, |
| | | | non-Hodgkin's lymphomas |
| | | Uracil mustard | Leukemia |
| | | Estramustine | Solid Tumors |
| | Ethylenimines and | Mitomycin C | Colorectal, ocular |
| | Methylmelamines | AZQ | Primary brain tumors |
| | | Thiotepa | Bladder, breast, ovary |
| | Alkyl Sulfonates | Busulfan, Hepsulfam | Chronic myelogenous leukemia |
| | Nitrosoureas | Carmustine | Hodgkin's disease, non-Hodgkin's |
| | | | lymphomas, primary brain tumors, multiple |
| | | | myeloma, malignant melanoma |
| | | Lomustine | Hodgkin's disease, non-Hodgkin's |
| | | | lymphomas, primary brain tumors, small- |
| | | | cell lung |
| | | Semustine | Primary brain tumors, stomach, colon |
| | | Streptozocin | Malignant pancreatic insulinama, |
| | | | malignant carcinoid |
| | Triazines | Dacarbazine | Malignant melanoma, Hodgkin's disease, |
| | | | soft-tissue sarcomas |
| | Platinum | Cisplatin, Carboplatin | Testis, ovary, bladder, head and neck, |
| | Complexes | | lung, thyroid, cervix, endometrium, |
| | | | neuroblastoma, osteogenic sarcoma |
| | Methyl Hydrazine | Procarbazine | Hodgkin's disease |
| | Derivative | | |
| Antimetabolites | Folic Acid Antagonists | Methotrexate, Trimetrexate | Acute lymphocytic leukemia, chorio- |
| | | | carcinoma, mycosis fungoides, breast, |
| | | | head and neck, lung, osteogenic sarcoma |
| | Pyrimidine Antagonists | Fluouracil, Floxuridine | Breast, colon, stomach, pancreas, ovary, |
| | | | head and neck, urinary bladder, skin, |
| | | | adenocarcinomas |
| | | Cytarabine | Acute myelogenous and acute |
| | | | lymphocytic leukemias |
| | | Fludarabine Phosphate | Lymphoproliferative disease |
| | | Capecitabine | Breast, renal cell, prostate |
| | | Azacitidine | acute leukemias |
| | Purine Antagonists | Thioguanine | Acute myelogenous, acute lymphocytic |
| | | | and chronic myelogenous leukemias |
| | | Mercaptopurine | Acute lymphocytic, acute myelogenous |
| | | | and chronic myelogenous leukemias |
| | | Allopurine | leukemias |
| | | Cladribine | Hairy cell leukemia |
| | | Gemcitabine | Pancreatic, soft tissue carcinomas |
| | | Pentostatin | Hairy cell leukemia, mycosis fungoides; |
| | | | chronic lymphocytic leukemia |
| Antimitotic Agents | | Vinblastine | Hodgkin's disease, non-Hodgkin's |
| | | | lymphomas, breast, testis |
| | | Vincristine | Acute lymphocytic leukemia, neuro- |
| | | | blastoma, Wilms' tumor, rhabdo- |
| | | | myosarcoma, Hodgkin's disease, non- |
| | | | Hodgkin's lymphomas, small-cell lung |
| DNA Topoisomerase II | | Etoposide, Teniposide | Testis, small-cell lung, oat-cell lung, |
| Inhibitors | | | breast, Hodgkin's disease, non-Hodgkin's |
| | | | lymphomas, acute myelogenous leukemia, |
| | | | Kaposi's sarcoma |
| DNA Topoisomerase I Inhibitors | | Topotecan, Irinotecan, | Ovarian, colorectal |
| | | Camptothecin, 9-Amino- | |
| | | camptothecin | |
| Taxanes | | Paclitaxel, Docetaxel | Breast |
| DNA Intercalators | | Daunorubicin | Acute myelogenous and acute lymphocytic |
| | | | leukemias |
| | | Doxorubicin | Ewing's sarcoma, osteosarcoma, rhabdo- |
| | | | myosarcomas, Hodgkin's disease, non- |
| | | | Hodgkin's lymphomas, acute leukemias, |
| | | | multiple myeloma, breast, genitourinary, |
| | | | thyroid, lung, ovarian, endometrial, |
| | | | testicular, stomach, neuroblastoma |
| | | Dactinomycin | Choriocarcinoma, Wilms' tumor, rhabdo- |
| | | | myosarcoma, testis, Kaposi's sarcoma |
| | | Idarubincin | Acute myeloid leukemia |
| | | Plicamycin | Testicular cancer |
| | | Mitomycin | Squamous sell carcinomas, small bladder |
| | | | papillomas, adenocarcinomas, pancreas, |
| | | | lung, colon, stomach, cervix, breast, head |
| | | | and neck |
| | | Amsacrine | Acute myelogenous leukemia, ovarian |
| | | | cancer, lymphomas |
| | | Bleomycin | Testicular, head and neck, skin, esophagus, |
| | | | squamous cell, colorectal, lung, |
| | | | genitourinary tract, cervix, ovarian, |
| | | | breast, Hodgkin's disease, non-Hodgkin's |
| | | | lymphomas |
| Hormonal | Aromatase | Aminoglutethimide, | Breast |
| Agents | Inhibitors | Anastrozole | |
| | 5-alpha-Reductase | Finasteride, Ketoconazole | Prostate |
| | Inhibitors | | |
| | Estrogen and | Tamoxifen | Breast |
| | Androgen | Flutamide | Prostate |
| | Inhibitors | | |
| | Gonadotropin | Leuprolide, Goserelin | Prostate |
| | Releasing | | |
| | Hormone Agonists | | |
| Tyrosine | ABL Inhibitors | Gleevec ™ (Novartis) | Chronic myelogenous leukemia or acute |
| Kinase Inhibitors | | | lymphoblastic leukemia |
| | PDGFR Inhibitors | Leflunomide (Pharmacia), | Gastrointestinal stromal tumor, small cell |
| | | SU5416 (Pharmacia), SU6668 | lung cancer, glioblastoma multiforme, |
| | | (Pharmacia), PTK787 | and prostate cancer |
| | | (Novartis) | |
| | EGFR Inhibitors | Iressa ™ (AstraZeneca), Tarceva ™, | Non-small-cell lung cancer, breast cancer, |
| | | (Oncogene Science), | ovarian cancer, bladder cancer, prostate |
| | | trastuzumab (Genentech), | cancer, salivary gland cancer, pancreatic |
| | | Erbitux ™ (ImClone), PKI166 | cancer, endometrial cancer, colorectal |
| | | (Novartis), GW2016 (Glaxo- | cancer, kidney cancer, head and neck |
| | | SmithKline), EKB-509 | cancer, glioblastoma multiforme |
| | | (Wyeth), EKB-569 (Wyeth), | |
| | | MDX-H210 (Medarex), 2C4 | |
| | | (Genentech), MDX-447 | |
| | | (Medarex), ABX-EGF | |
| | | (Abgenix), CI-1033 (Pfizer) | |
| | VEGFR Inhibitors | Avastin ™ (Genentech), IMC- | Any solid tumor |
| | | 1C11 (ImClone), ZD4190 | |
| | | (AstraZeneca), ZD6474 | |
| | | (AstraZeneca ) | |
| | Trk Inhibitors | CEP-701 (Cephalon), CEP- | Prostate cancer, pancreatic cancer |
| | | 751 (Cephalon) | |
| | Flt-3 Inhibitors | MLN518 (Millennium), | Acute myeloid leukemia |
| | | PKC412 (Novartis) | |

| | | |
|---|---|---|
| Retinoic Acid Derivatives | 13-cis-retinoic acid, iso- | Acute promyelocytic leukemia, head and |
| | tretinoin, retinyl palmitate, 4- | neck squamous cell carcinoma |
| | (hydroxycarbophenyl) | |
| | retinamide | |
| Hypoxia-Selective Cytoxins | Misonidazole | Head and neck |
| | Nitracrine | Breast |
| Miscellaneous Agents | Mitoxantrone | Acute myelogenous leukemia non- |
| | | Hodgkin's lymphoma's, breast |
| | Hydroxyurea | Chronic myelogenous leukemia, poly- |
| | | cythemia vera, essential thrombocytosis, |
| | | malignant melanoma |
| | L-Asparaginase | Acute lymphocytic leukemia |
| | Interferon alfa | Hairy cell leukemia., Kaposi's sarcoma, |
| | | melanoma, carcinoid, renal cell, ovary, |
| | | bladder, non-Hodgkin's lymphomas, |
| | | mycosis fungoides, multiple myeloma, |
| | | chronic myelogenous leukemia |
| | Rapamycin, CCI-779 | Glioblastoma multiforme, renal cell |
| | | carcinoma |
| | Mitotane | Adrenal carcinoma |

In the methods of the present invention, the dosage and frequency of administration of the compound of the invention and additional antiproliferative agent(s) can be controlled independently. For example, one compound may be administered orally three times per day, while the second compound may be administered intravenously once per day. The compounds may also be formulated together such that one administration delivers both compounds.

The exemplary dosage of the compound and additional antiproliferative agent(s) to be administered will depend on such variables as the type and extent of the disorder, the overall health status of the patient, the therapeutic index of the selected antiproliferative agent(s), and route of administration. Standard clinical trials may be used to optimize the dose and dosing frequency for any particular combination.

Administration

The invention features compositions and methods that can be used to modulate the effects of local and systemic hypoxic events. The compounds of the invention can be formulated with a pharmaceutically acceptable excipient prior to administration. These pharmaceutical compositions can be prepared according to the customary methods, using one or more pharmaceutically acceptable adjuvants or excipients. The adjuvants comprise, without limitation, diluents, sterile aqueous media, and various non-toxic organic solvents. Acceptable carriers or diluents for therapeutic use are well known in the pharmaceutical field, and are described, for example, in Remington: The Science and Practice of Pharmacy (20th ed.), ed. A. R. Gennaro, Lippincott Williams & Wilkins, 2000, Philadelphia, and Encyclopedia of Pharmaceutical Technology, eds. J. Swarbrick and J. C. Boylan, 1988-1999, Marcel Dekker, New York. The compositions may be presented in the form of tablets, pills, granules, powders, aqueous solutions or suspensions, injectable solutions, elixirs, or syrups, and the compositions may optionally contain one or more agents chosen from the group comprising sweeteners, flavorings, colorings, and stabilizers in order to obtain pharmaceutically acceptable preparations.

Dosage levels of active ingredients in the pharmaceutical compositions of the invention may be varied to obtain an amount of the active compound(s) that achieves the desired therapeutic response for a particular patient, composition, and mode of administration. The selected dosage level depends upon the activity of the particular compound, the route of administration, the severity of the condition being treated, and the condition and prior medical history of the patient being treated. For adults, the doses are generally from about 0.01 to about 100 mg/kg, desirably about 0.1 to about 1 mg/kg body weight per day by inhalation, from about 0.01 to about 100 mg/kg, desirably 0.1 to 70 mg/kg, more desirably 0.5 to 10 mg/kg body weight per day by oral administration, and from about 0.01 to about 50 mg/kg, desirably 0.1 to 1 mg/kg body weight per day by intravenous administration. Doses are determined for each particular case using standard methods in accordance with factors unique to the patient, including age, weight, general state of health, and other factors which can influence the efficacy of the compound(s) of the invention.

The compound of the invention can be administered orally, parenterally by intravenous injection, transdermally, by pulmonary inhalation, by intravaginal or intrarectal insertion, by subcutaneous implantation, intramuscular injection or by injection directly into an affected tissue; as for example by injection into a tumor site. In some instances the materials may be applied topically at the time surgery is carried out. In another instance the topical administration may be ophthalmic, with direct application of the therapeutic composition to the eye.

For example, the compound of the invention can be administered to a patient by using an osmotic pump, such as the Alzet® Model 2002 osmotic pump. Osmotic pumps provides continuous delivery of test agents, thereby eliminating the need for frequent, round-the-clock injections. With sizes small enough even for use in mice or young rats, these implantable pumps have proven invaluable in predictably sustaining compounds at therapeutic levels, avoiding potentially toxic or misleading side effects.

Alternatively, the compound of the invention can be administered to a patient's eye in a controlled manner. There are numerous devices and methods for delivering drugs to the eye. For example, U.S. Pat. No. 6,331,313 describes various controlled-release devices which are biocompatible and can be implanted into the eye. The devices described therein have a core comprising a drug and a polymeric outer layer which is substantially impermeable to the entrance of an environmental fluid and substantially impermeable to the release of the drug during a delivery period, and drug release is effected through an orifice in the outer layer. These devices have an orifice area of less than 10% of the total surface area of the device and can be used to deliver a variety of drugs with varying degrees of solubility and or molecular weight. Methods are also provided for using these drug delivery devices. The biocompatible, implantable ocular controlled-release drug delivery device is sized for implantation within an eye for continuously delivering a drug within the eye for a period of at least several weeks. Such device comprises a polymeric outer layer that is substantially impermeable to the drug and ocular fluids, and covers a core comprising a drug that dissolves in ocular fluids, wherein the outer layer has one or more orifices through which ocular fluids may pass to contact the core and dissolve drug, and the dissolved drug may pass to the exterior of the device. The orifices in total may have an area less than one percent of the total surface area of the device, and the rate of release of the drug is determined solely by the composition of the core and the total surface area of the one or more orifices relative to the total surface area of the device. Other examples ocular implant methods and devices, and related improvements for drug delivery in the eye are described in U.S. Pat. No. 5,824,072, U.S. Pat. No. 5,766,242, U.S. Pat. No. 5,632,984, U.S. Pat. No. 5,443,505, and U.S. Pat. No. 5,902,598; US 2004/0175410-A1, US 2004/0151754-A1, US 2004/0022853-A1, US 2003/0203030-A1; and WO 95/13765-A1, WO 01/30323-A2, WO 02/02076-A2, WO 02/43785-A2, and WO 2004/026106-A2.

For certain applications the compound of the invention may be need to be delivered locally. In such cases, various known methods in the art may be used to achieve limited local delivery without causing undesirable systemic side effects. To just name a few, WO 03/066130-A2 (MIT, entire contents incorporated herein by reference) discloses a transdermal delivery system including a drug formulated with a transport chaperone moiety that reversibly associates with the drug. The chaperone moiety is associated with the drug in the formulation so as to enhance transport of the drug across dermal tissue and releasing the drug after crossing said dermal tissue. The application also provides a micro-emulsion system for transdermal delivery of a steroidal HIF-1 modulator, which system solubilizes both hydrophilic and hydrophobic components. For instance, the microemulsion can be a cosolvent system including a lipophilic solvent and an organic solvent. Exemplary cosolvents are N-methylpyrrolidone (NMP) and isopropyl myristate (IPM).

WO 02/087586-A1 (Control Delivery Systems, Inc.) discloses a sustained release system that includes a polymer and a prodrug having a solubility less than about 1 mg/ml dispersed in the polymer. Advantageously, the polymer is permeable to the prodrug and may be non-release rate limiting with respect to the rate of release of the prodrug from the polymer. This permits improved drug delivery within a body in the vicinity of a surgery via sustained release rate kinetics over a prolonged period of time, while not requiring complicated manufacturing processes.

The materials are formulated to suit the desired route of administration. The formulation may comprise suitable excipients include pharmaceutically acceptable buffers, stabilizers, local anesthetics, and the like that are well known in the art. For parenteral administration, an exemplary formulation may be a sterile solution or suspension; for oral dosage, a syrup, tablet or palatable solution; for topical application, a lotion, cream, spray or ointment; for administration by inhalation, a microcrystalline powder or a solution suitable for nebulization; for intravaginal or intrarectal administration, pessaries, suppositories, creams or foams.

Compounds

Compounds of the invention include compounds where the linker of BP228 has been extended to 3-5 carbon atoms:

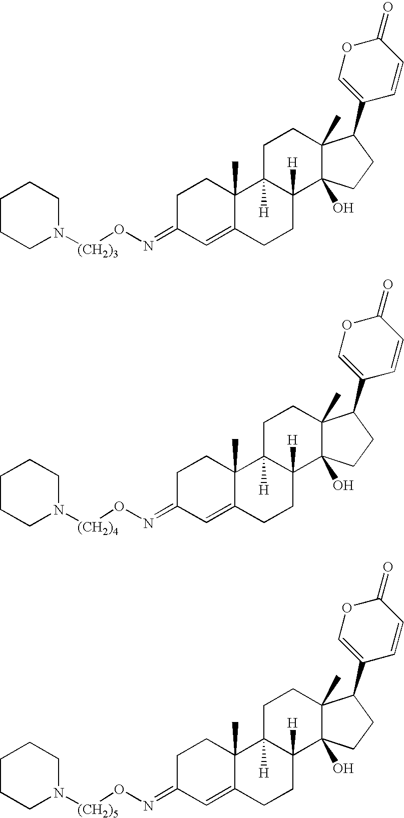

compounds where the piperidine has been substituted with methyl:

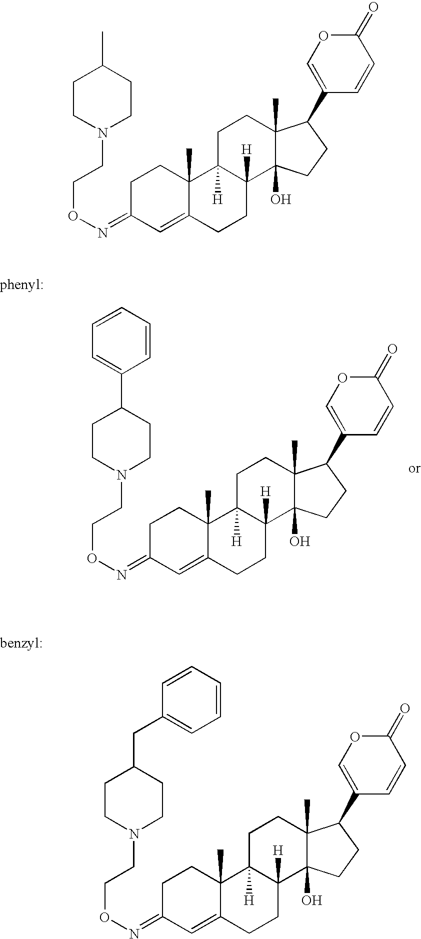

and replacement of piperidine by morpholine:

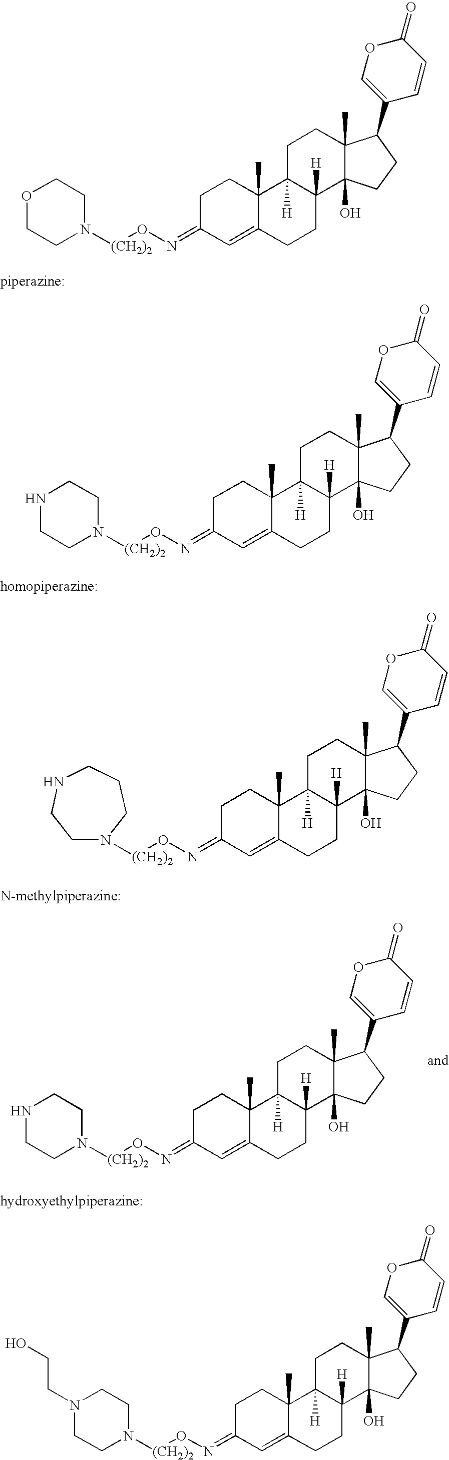

These have been shown here in the E configuration, though Z configuration is also possible.

Synthesis

Many 3-hydroxy bufadienolide steroids have been previously described, such as, for example, those described by Kamano et al., in J. Med. Chem. 45: 5440-5447, 2002; Kamano et al., in J. Nat. Prod. 65: 1001-1005, 2002; Nogawa et al., in J. Nat. Prod. 64: 1148-1152, 2001; and Qu et al., J. Steroid Biochem. Mol. Biol. 91: 87-98.

In addition, several different routes to the preparation of bufadienolides have been described in the art, including Soncheimer et al., J. Am. Chem. Soc. 91: 1228-1230, 1969; Stache et al., Tetrahedron Lett. 35: 3033-3038, 1969; Pettit et al., Can. J. Chem. 47: 2511, 1969; Pettit et al., J. Org. Chem. 35: 1367-9, 1970; Tsay et al., Heterocycles 12: 1397-1402, 1979; Sen et al., J. Chem. Soc. Chem. Comm. 66: 1213-1214, 1982; Wiesner et al., Helv. Chim. Acta 66: 2632-2641, 1983; Weisner & Tsai, Pure and Appl. Chem. 53: 799-810, 1986, and U.S. Pat. Nos. 4,001,402; 4,102,884; 4,175,078; 4,242,332; and 4,380,624.

### Examples

The following examples are put forth so as to provide those of ordinary skill in the art with a complete disclosure and description of how the methods and compounds claimed herein are performed, made and evaluated, and are intended to be purely exemplary of the invention and are not intended to limit the scope of what the inventors regard as their invention.

The comparative HIF-1-modulating compounds used in following studies are referred to as BNC1, BNC3 and BNC4.

BNC1 is ouabain or G-Strophanthin (STRODIVAL®), which has been used for treating myocardial infarction. It is a colorless crystal with predicted IC₅₀ of about 0.06-0.35 μg/mL and max. plasma concentration of about 0.03 μg/mL. According to the literature, its plasma half-life in human is about 20 hours, with a range of between 5-50 hours. Its common formulation is injectable. The typical dose for current indication (i.v.) is about 0.25 mg, up to 0.5 mg/day.

BNC3 is bicalutamide (CASODEX®), an androgen response inhibitor.

BNC4 is proscillaridin (TALUSIN®), which has been approved for treating chronic cardiac insufficiency in Europe. It is a colorless crystal with predicted IC₅₀ of about 0.01-0.06 μg/mL and max. plasma concentration of about 0.1 μg/mL. According to the literature, its plasma half-life in human is about 40 hours. Its common available formulation is a tablet of 0.25 or 0.5 mg. The typical dose for current indication (p.o.) is about 1.5 mg /day.

### Example 1

### Cardiac Glycoside Compounds Inhibits HIF-1α Expression

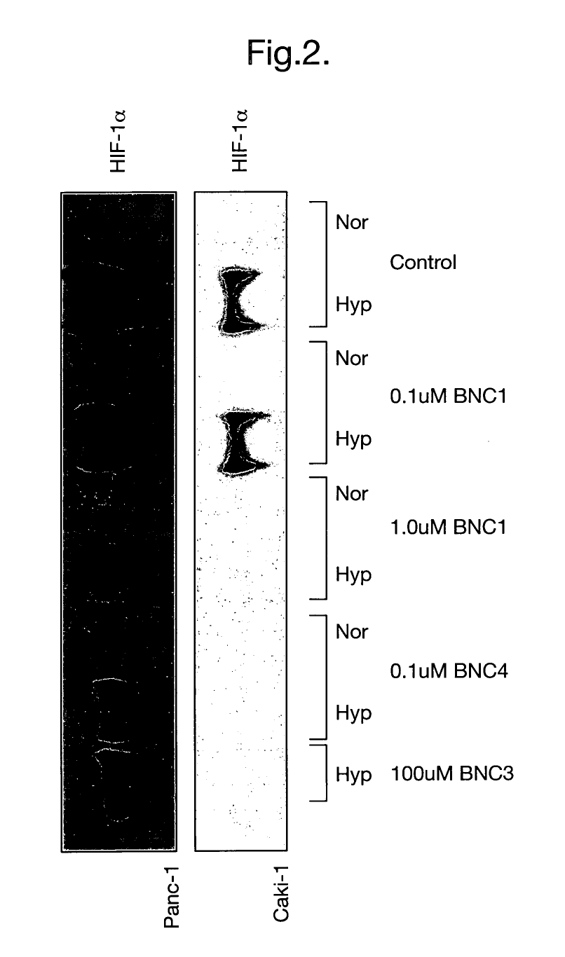

The ability of BNC1 and BNC4 to inhibit hypoxia-mediated HIF1α induction in human tumor cells was investigated. FIG. 2 shows the result of immunoblotting for HIF-1α expression in Caki-1 (renal cancer) or Panc-1 (pancreatic cancer) cells treated with BNC1, BNC3 or BNC4 under hypoxia. The results indicate that BNC4 is about 10 times more potent than BNC 1 in inhibiting HIF-1α expression.

### Example 2

### BNC4 Inhibits HIF-1α Induced Under Normoxia by PHD Inhibitor

To study the mechanism of BNC4 inhibition of HIF-la, the ability of BNC1 or BNC4 to inhibit HIF-1α expression induced by a prolyl-4-hydroxylase domain (PHD) inhibitor, L-mimosone, was investigated under normoxia condition.

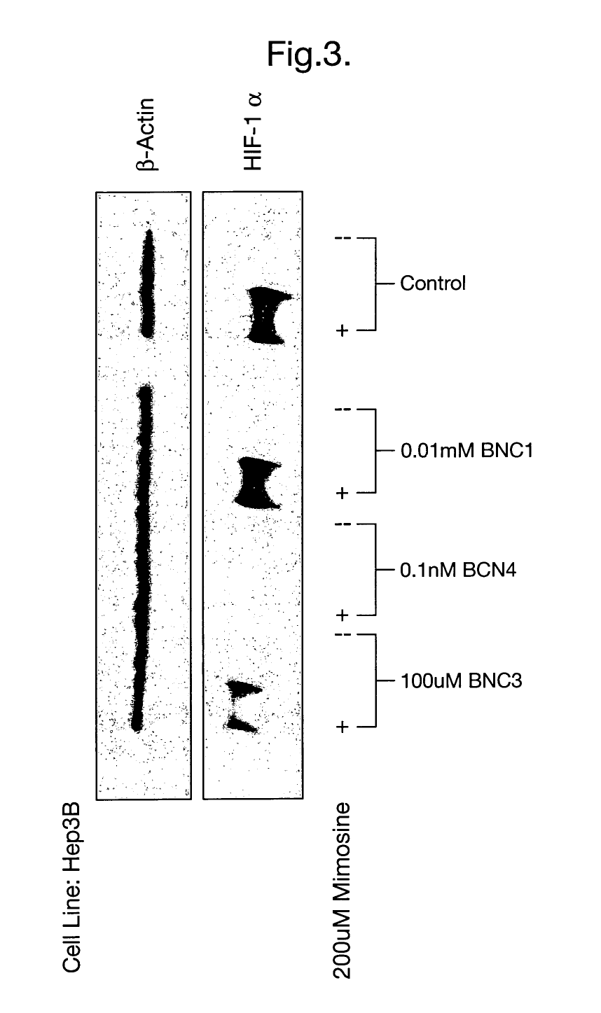

In the experiment represented in FIG. 3, Hep3B cells were grown under normoxia, but were also treated as indicated with 200 μM L-mimosone for 18 hours in the presence or absence of BNC1, BNC3 or BNC4. Abundance of HIF1α and β-actin was determined by Western blotting.

The results indicate that L-mimosone induced HIF-1α accumulation under normoxia condition, and addition of BNC4 eliminated HIF-1α accumulation by L-mimosone. At the low concentration tested, BNC1 did not appear to have an effect on HIF-1α accumulation in this experiment. The fact that BNC4 can inhibit HIF-1α induced under normoxia by PHD inhibitor indicates that the site of action by BNC4 probably lies downstream of prolyl-hydroxylation.

### Example 3

### Preparation of 3-Oximethers of Scillarenin

Synthesis of Scillarenin

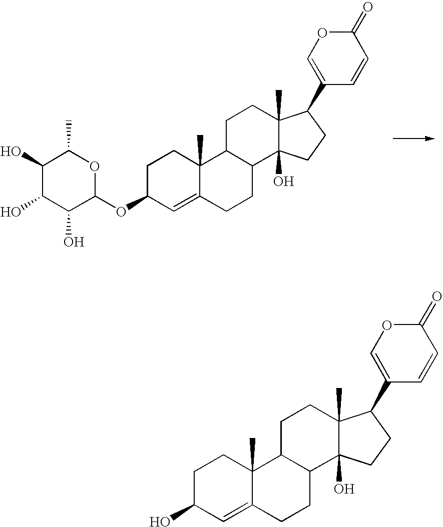

A solution (partial suspension) of proscillaridin (66.3 mg, 0.125 mmol) and naringinase (23.2 mg) in EtOH (1.25 mL)-0.02 M acetate buffer (pH 4.0, 3.75 mL) was incubated at 40° C. for 6.5 h. After addition of EtOH (30 mL), the whole mixture was concentrated under reduced pressure. The resulting residue was purified by column chromatography (SiO₂, 10 g, n-hexanes-EtOAc (1:1)) to furnish scillarenin (48 mg).

Synthesis of Scillarenon

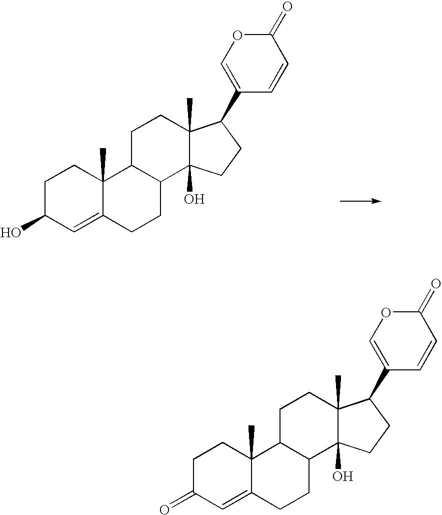

700 mg (1.82 mmole) of scillarenin was dissolved in 30 mL of dry dichloromethane and 1.4 g of powdered molecular sieve and 1.57 g (7.28 mmole) of pyridinium chlorochromate were added. The mixture was stirred under a nitrogen atmosphere at room temperature overnight. The dark mixture was filtered through a pad of Celite and concentrated. The crude mixture was purified by flash chromatography to yield 604 mg (86%) of the desired ketone as a colorless solid.

Synthesis of Hydroxylamine Intermediate

Scillarenin 3-oximethers derivatives can be prepared as described below in Schemes 1, 2 and 3.

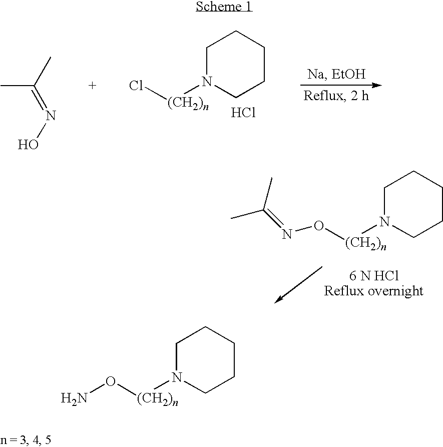

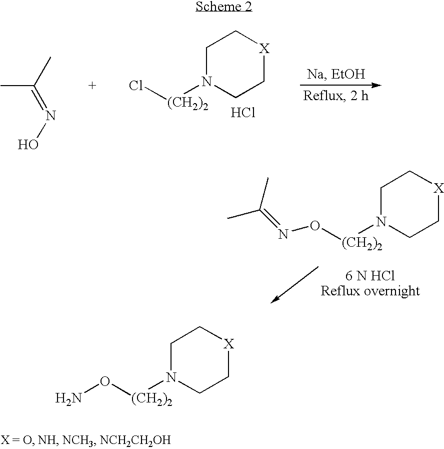

Protection may be used when X═NCH₂CH₂OH. Any standard acid-labile group for protecting alcohols may be used. See for example “Protective groups in organic synthesis” 3rd Ed, Theodora W Greene and Peter G. Wuts, published by John Wiley, ISBN 0 471 16019 9. It might also be protected as a benzyl, methoxymethyl (MOM) or silyl ether.

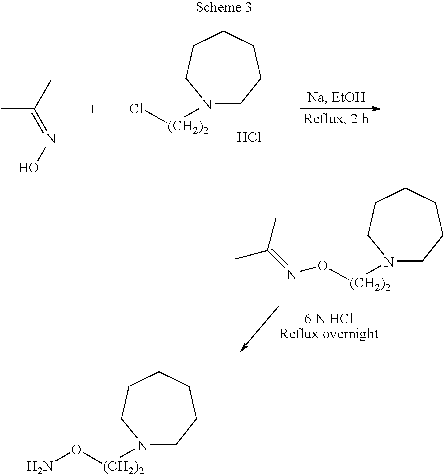

Synthesis of Scillarenin 3-Oximethers

Scillarenin 3-oximethers derivatives can be prepared as described below in Scheme 4. Typically this method gives an equilibrium mixture of the E and Z isomers. The E and Z isomers appear to interconvert under normal experimental conditions without decomposing, so isolation of individual geometric isomers has not been possible. As any single geometrical isomer would rapidly revert to an equilibrium mixture under physiological conditions, isolation is not necessary.

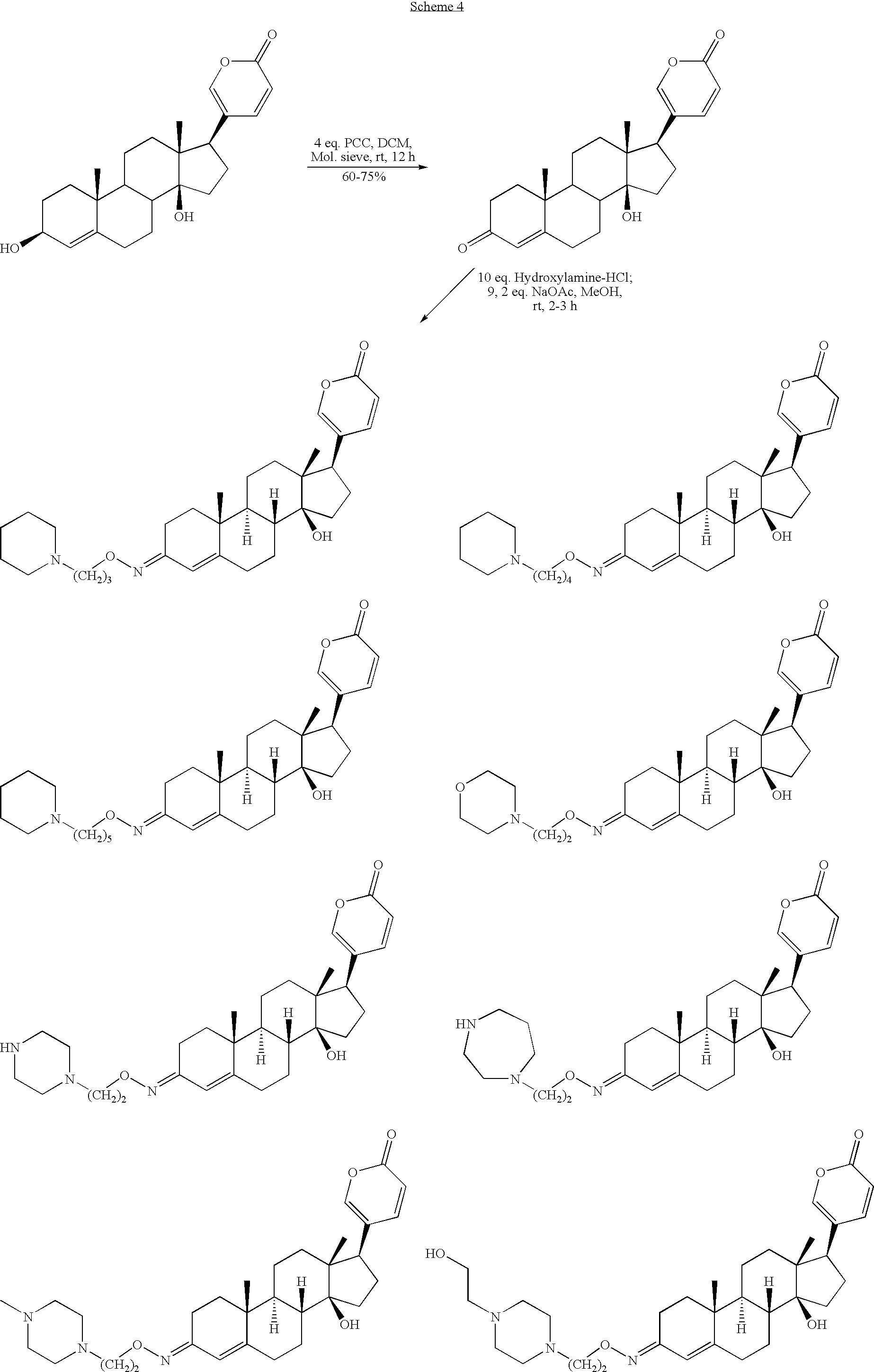

### Example 4

### General Procedures

### 1. Preparation of Hydroxyl Amines

Method A:

A solution of the corresponding alkyl amine-substituted N-hydroxy-5-norbornene-2,3-dicarboximide derivatives (2.0 mmole) [prepared according to Bioorg. Med. Chem. 1998, 6, 811] in 3.34 ml (10 eq.) 6 N HCl was refluxed for 30 min. After cooling down the mixture was evaporated to dryness and the solid residue was recrystallized from an appropriate solvent to yield the desired hydroxyl amine×2HCl salts in pure form.

Method B:

A solution of the corresponding alkyl amine-substituted N-hydroxy-5-norbornene-2,3-dicarboximide derivatives (3.5 mmole) in 6 ml 95% EtOH and hydrazine hydrate (0.385 ml, 7.9 mmole) was heated for 3×6 min at 120° C. in a microwave oven (Biotage, Initiator). The reaction mixture is cooled to 0° C. and the precipitated solid was filtered and washed with cold EtOH. The filtrate was evaporated to dryness and taken up in 50% aqueous NaOH solution. Extraction of the aqueous layer with diethyl ether, drying of the combined organic extract and subsequent concentration yielded the free hydroxyl amines pure enough for the next step.

### 2-(Aminooxy)ethyl morpholine dihydrochloride

The material was prepared according to method A and recrystallized from EtOH/water 9+1 to give 333 mg (55%) of a colorless solid. ¹H-NMR (4-MeOH): δ 3.45 (br m, 4H), 3.64 (t, 2H, J=4.8 Hz), 4.02 (br m, 4H), 4.53 (t, 2H, J=4.8 Hz).

### 3-(Aminooxy)propyl piperidine dihydrochloride

The material was prepared according to method A and recrystallized from EtOH to give 288 mg (62%) of a colorless solid. ¹H-NMR (d₄-MeOH): 6 1.58 (m, 1H), 1.84-2.04 (m, 5H), 2.23 (m, 2H), 3.01 (dt, 2H, J=2.8, 12.4 Hz), 3.27 (t, 2H, J=8.0 Hz), 3.60 (d, 2H, J=12.4 Hz), 4.23 (t, 2H, J=5.6 Hz).

### 4-(Aminooxy)butyl piperidine dihydrochloride

The material was prepared according to method A and recrystallized from EtOH/Ether 1+1 to give 258 mg (59%) of a colorless solid. ¹H-NMR (d₄-MeOH): δ 1.58 (m, 1H), 1.78-2.04 (m, 9H), 2.98 (dt, 2H, J=2.4, 12.8 Hz), 3.17 (t, 2H, J=8.4 Hz), 3.58 (d, 2H, J=11.2 Hz), 4.14 (t, 2H, J=6.0 Hz).

### 5-(Aminooxy)pentyl piperidine

The material was prepared according to method B to give 556 mg (85%) of a colorless oil. ¹H-NMR (CDCl₃): δ 1.25-1.65 (m, 12H), 2.26 (t, 2H, J=7.6 Hz), 2.35 (br s, 4H), 3.65 (t, 2H, J=7.2 Hz), 5.33 (br s, 2H).

### 2-(Aminooxy)ethyl-(4-benzyl) piperidine dihydrochloride

The material was prepared according to method A and recrystallized from EtOH to give 600 mg (60%) of a colorless solid. ¹H-NMR (d₄-MeOH): 8 1.55-1.72 (m, 2H), 1.95 (m, 3H), 2.68 (m, 2H), 3.06 (t, 2H, J=12.0 Hz), 3.54 (br m, 2H), 3.64 (m, 2H), 4.50 (t, 2H, J=4.8 Hz), 7.24 (m, 3H), 7.33 (m, 2H).

### 2. Preparation of Oxime Ethers

Method A:

To a solution of 25 mg (0.065 mmole) scillarinone in 2 ml MeOH 98 mg (1.2 mmole) NaOAc and 0.65 mmole (10 eq.) of the corresponding hydroxylamine dihydrochloride was added and the mixture stirred at room temperature for 1 h. The mixture was diluted with ethyl acetate and washed with saturated NaHCO₃-solution and brine. The aqueous phase was extracted with ethyl acetate twice, dried over Na₂SO₄ and concentrated under reduced pressure. The residue was purified by flash chromatography.

Method B:

To a solution of 25 mg (0.065 mmole) scillarinone in 2 ml MeOH 0.26 mmole (4 eq.) of the corresponding hydroxyl amine and 130 μl acetic acid was added and the mixture stirred at room temperature for 1 h. The mixture was diluted with ethyl acetate and washed with saturated NaHCO₃-solution and brine. The aqueous phase was extracted with ethyl acetate twice, dried over Na₂SO₄ and concentrated under reduced pressure. The residue was purified by flash chromatography.

### Compound 1: 3-[2-(Morpholinoethoxy)imino]-scillarinone, BP245

The material was prepared according to method A to give 17 mg (51%) of a colorless solid.

### Compound 2: 3-[3-(Piperidinopropoxy)imino]-scillarinone, BP246

The material was prepared according to method A to give 15 mg (44%) of a colorless solid.

### Compound 3: 3-[4-(Piperidinobutoxy)imino]-scillarinone, BP247

The material was prepared according to method A to give 23 mg (66%) of a colorless solid.

### Compound 4: 3-[5-(Piperidinopentoxy)imino]-scillarinone, BP248

The material was prepared according to method B to give 18 mg (50%) of a colorless solid.

### Compound 5: 3-[2-(4-Benzylpiperidinoethoxy)imino]-scillarinone, BP249

The material was prepared according to method A to give 17 mg (36%) of a colorless solid.

3. Spectroscopic data

Compound 1

| Compound ID | BP245 |
|---|---|
| Name | 5-((10R,13R,14S,17R)-14-hydroxy-10,13-dimethyl-3-(2-mor- |
| | pholinoethoxyimino)-2,3,6,7,8,9,10,11,12,13,14,15,16,17- |
| | tetradecahydro-1H-cyclopenta[α]-phenanthren-17-yl)- |
| | 2H-pyran-2-one |
| Molecular formula | C₃₀H₄₂N₂O₅ |
| Molecular weight | 510.68 |
| [g/mol] | |
| Purity (LC/MS- | 100% |
| ELSD) | |
| Comment | 3:2-mixture of E/Z-Isomers |
| Structure [numbering scheme by analogy to scillarenone] | 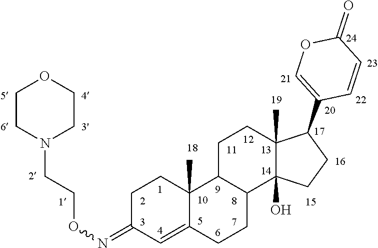 |

### LC/MS Assignment:

| | |
|---|---|
| (+)-ESI | (−)-ESI |

| m/z | Interpretation | m/z | Interpretation |
|---|---|---|---|
| 511 | [M + H]⁺ | 555 | [M + HCOO]⁻ |

Assignment of the ¹H- and ¹³C-NMR-Signals* (based on HH-COSY, HSQC, and HMBC Experiments)

| | | | |
|---|---|---|---|
| | | | J [Hz]/ |
| Position | ¹³C [ppm] | ¹H [ppm] | Int. |

| 1 | 35.3 | 1.45 | m | |
|---|---|---|---|---|
| | | 2.00 | m | |
| 2 | 19.8 | 2.17 | m | |
| | | 3.05 | m | |
| 3 | 155.4 | — | | |
| 4 | 118.1 (111.7)** | 5.89 s (6.48 s)** | | |
| 5 | 157.7 | — | | |
| 6 | 33.2 | 2.41 | m | (2H) |
| 7 | 28.2 | 1.28 | m | |
| | | 2.17 | m | |
| 8 | 43.0 | 1.68 | m | |
| 9 | 49.9 | 1.22 | m | |
| 10 | 37.9 | — | | |
| 11 | 21.9 | 1.44 | m | |
| | | 1.62 | m | |
| 12 | 40.9 | 1.48 | m | |
| | | 1.62 | m | |
| 13 | 48.3 | — | | |
| 14 | 85.4 | — | | |
| 15 | 33.0 | 1.75 | m | |
| | | 2.05 | m | |
| 16 | 28.8 | 1.85 | m | |
| | | 2.27 | m | |
| 17 | 51.5 | 2.57 | m | |
| 18 | 18.3 (18.6)** | 1.14 s (1.18 s)** | | (3H) |
| | | | | (3H) |
| 19 | 16.7 | 0.84 | s | (3H) |
| 20 | 122.7 | — | | |
| 21 | 148.7 | 7.33 | d | 1.9 |
| 22 | 147.0 | 7.92 | dd | 9.4; 1.9 |
| 23 | 115.8 | 6.37 | d | 9.4 |
| 24 | 162.8 | — | | |
| 1′ | 71.6 | 4.33 | m | (2H) |
| 2′ | 57.9 | 2.85 | m | (2H) |
| 3′/6′ | 53.2 | 2.70 | m | (4H) |
| 4′/5′ | 67.0 | 3.85 | m | (4H) |

*Solvent: CDCl₃ (δ_{C} = 77.5 ppm; δ_{H} = 7.36 ppm)
**Signal of the minor stereoisomer

Compound 2

| Compound ID | BP246 |
|---|---|
| Name | 5-((10R,13R,14S,17R)-14-hydroxy-10,13-dimethyl-3-(3-(piperidin-1-yl) |
| | propoxyimino)-2,3,6,7,8,9,10,11,12,13,14,15,16,17-tetradecahydro-1H- |
| | cyclopenta[α]phenanthren-17-yl)-2H-pyran-2-one |
| Molecular formula | C₃₂H₄₆N₂O₄ |
| Molecular weight | 522.73 |
| [g/mol] | |
| Purity (LC/MS- | 100% |
| ELSD) | |
| Comment | 2:1-mixture of E/Z-Isomers |
| Structure | 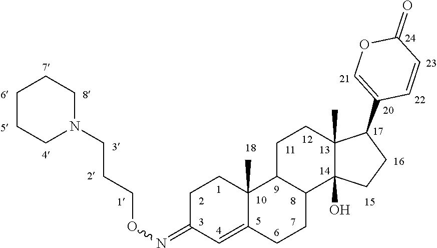 |

### LC/MS Assignment:

| | |
|---|---|
| (+)-ESI | (−)-ESI |

| m/z | Interpretation | m/z | Interpretation |
|---|---|---|---|
| 523 | [M + H]⁺ | 567 | [M + HCOO]⁻ |

### Assignment of the ¹H- and ¹³C-NMR-Signals* (Based on HH-COSY, HSQC, and HMBC Experiments)

| | | | |
|---|---|---|---|
| | | | J [Hz]/ |
| Position | ¹³C [ppm] | ¹H [ppm] | Int. |

| 1 | 35.1 | 1.47 | m | |
|---|---|---|---|---|
| | | 1.97 | m | |
| 2 | 19.7 | 2.18 | m | |
| | | 3.04 | m | |
| 3 | 156.3 | — | | |
| 4 | 118.0 (111.6)** | 5.87 s (6.48 s)** | | |
| 5 | 154.4 | — | | |
| 6 | 33.2 | 2.40 | m | (2H) |
| 7 | 28.2 | 1.29 | m | |
| | | 2.18 | m | |
| 8 | 42.9 | 1.68 | m | |
| 9 | 50.0 | 1.22 | m | |
| 10 | 38.3 | — | | |
| 11 | 21.9 | 1.43 | m | |
| | | 1.60 | m | |
| 12 | 40.7 | 1.47 | m | |
| | | 1.64 | m | |
| 13 | 48.4 | — | | |
| 14 | 85.3 | — | | |
| 15 | 33.1 | 1.74 | m | |
| | | 2.04 | m | |
| 16 | 28.7 | 1.84 | m | |
| | | 2.27 | m | |
| 17 | 51.4 | 2.57 | m | |
| 18 | 18.4 (18.6)** | 1.13 s (1.18 s)** | | (3H) |
| 19 | 16.8 | 0.84 | s | (3H) |
| 20 | 122.8 | — | | |
| 21 | 148.9 | 7.33 | d | 1.9 |
| 22 | 146.8 | 7.92 | dd | 9.4; 1.9 |
| 23 | 115.7 | 6.36 | d | 9.4 |
| 24 | 162.6 | — | | |
| 1′ | 71.9 | 4.17 | m | (2H) |
| 2′ | 25.9 | 2.10 | m | (2H) |
| 3′ | 56.1 | 2.72 | m | (2H) |
| 4′/8′ | 54.5 | 2.72 | m | (4H) |
| 5′/7′ | 25.3 | 1.84 | m | (4H) |
| 6′ | 24.0 | 1.61 | m | (2H) |

*Solvent: CDCl₃ (δ_{C} = 77.5 ppm; δ_{H} = 7.36 ppm)
**Signal of the minor stereoisomer

Compound 3

| Compound ID | BP247 |
|---|---|
| Name | 5-((10R,13R,14S,17R)-14-hydroxy-10,13-dimethyl-3-(4-(piperidin- |
| | 1-yl)butoxyimino)-2,3,6,7,8,9,10,11,12,13,14,15,16,17-tetradeca- |
| | hydro-1H-cyclopenta[α]phenanthren-17-yl)-2H-pyran-2-one |
| Molecular formula | C₃₃H₄₈N₂O₄ |
| Molecular weight | 536.76 |
| [g/mol] | |
| Purity (LC/MS-ELSD) | 100% |
| Comment | 2:1-mixture of E/Z-Isomers |
| Structure | 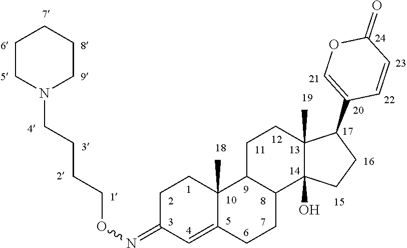 |

### LC/MS Assignment:

| | |
|---|---|
| (+)-ESI | (−)-ESI |

| m/z | Interpretation | m/z | Interpretation |
|---|---|---|---|
| 538 | [M + H]⁺ | 581 | [M + HCOO]⁻ |

### Assignment of the ¹H- and ¹³C-NMR-Signals* (Based on HH-COSY, HSQC, and HMBC Experiments)

| | | | |
|---|---|---|---|
| | | | J [Hz]/ |
| Position | ¹³C [ppm] | ¹H [ppm] | Int. |

| 1 | 35.2 | 1.46 | m | |
|---|---|---|---|---|
| | | 1.98 | m | |
| 2 | 19.8 | 2.18 | m | |
| | | 3.06 | m | |
| 3 | 156.0 | — | | |
| 4 | 118.1 (111.7)** | 5.88 s (6.50 s)** | | |
| 5 | 154.0 | — | | |
| 6 | 33.1 | 2.39 | m | (2H) |
| 7 | 28.1 | 1.27 | m | |
| | | 2.18 | m | |
| 8 | 42.9 | 1.69 | m | |
| 9 | 49.9 | 1.22 | m | |
| 10 | 38.3 | — | | |
| 11 | 22.0 | 1.43 | m | |
| | | 1.59 | m | |
| 12 | 40.8 | 1.47 | m | |
| | | 1.62 | m | |
| 13 | 48.4 | — | | |
| 14 | 85.4 | — | | |
| 15 | 33.0 | 1.70 | m | |
| | | 2.06 | m | |
| 16 | 28.7 | 1.84 | m | |
| | | 2.27 | m | |
| 17 | 51.4 | 2.55 | m | |
| 18 | 18.3 (18.5)** | 1.12 s (1.17 s)** | | (3H) |
| 19 | 16.8 | 0.83 | s | (3H) |
| 20 | 122.9 | — | | |
| 21 | 148.9 | 7.33 | d | 1.9 |
| 22 | 147.0 | 7.93 | dd | 9.4; 1.9 |
| 23 | 115.6 | 6.36 | d | 9.4 |
| 24 | 162.7 | — | | |
| 1′ | 73.7 | 4.14 | m | (2H) |
| 2′ | 27.7 | 1.75 | m | (2H) |
| 3′ | 23.3 | 1.75 | m | (2H) |
| 4′ | 59.2 | 2.53 | m | (2H) |
| 5′/9′ | 54.5 | 2.57 | m | (4H) |
| 6′/8′ | 25.6 | 1.76 | m | (4H) |
| 7′ | 24.3 | 1.56 | m | (2H) |

*Solvent: CDCl₃ (δ_{C} = 77.5 ppm; δ_{H} = 7.36 ppm)
**Signal of the minor stereoisomer

Compound 4

| Compound ID | BP248 |
|---|---|
| Name | 5-((10R,13R,14S,17R)-14-hydroxy-10,13-dimethyl-3-(5-(piperidin- |
| | 1-yl)pentyloxyimino)-2,3,6,7,8,9,10,11,12,13,14,15,16,17-tetradeca- |
| | hydro-1H-cyclopenta[α]pehanthren-17-yl)-2H-pyran-2-one |
| Molecular formula | C₃₄H₅₀N₂O₄ |
| Molecular weight [g/mol] | 550.79 |
| Purity (LC/MS-ELSD) | 100% |
| Comment | 3:2-mixture of E/Z-Isomers |
| Structure | 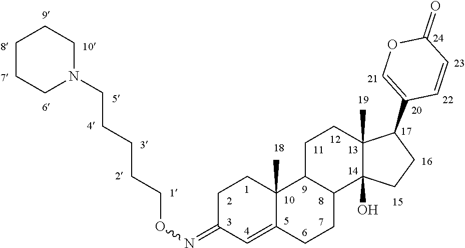 |

### LC/MS Assignment:

| | |
|---|---|
| (+)-ESI | (−)-ESI |

| m/z | Interpretation | m/z | Interpretation |
|---|---|---|---|
| 551 | [M + H]⁺ | 595 | [M + HCOO]⁻ |

### Assignment of the ¹H- and ¹³C-NMR-Signals* (Based on HH-COSY, HSQC, and HMBC Experiments)

| | | | |
|---|---|---|---|
| | | | J [Hz]/ |
| Position | ¹³C [ppm] | ¹H [ppm] | Int. |

| 1 | 35.3 | 1.48 | m | |
|---|---|---|---|---|
| | | 1.99 | m | |
| 2 | 19.7 | 2.19 | m | |
| | | 3.05 | m | |
| 3 | 156.1 | — | | |
| 4 | 118.0 (111.5)** | 5.88 s (6.49 s)** | | |
| 5 | 154.1 | — | | |
| 6 | 33.2 | 2.41 | m | (2H) |
| 7 | 28.0 | 1.28 | m | |
| | | 2.18 | m | |
| 8 | 42.9 | 1.69 | m | |
| 9 | 49.9 | 1.23 | m | |
| 10 | 38.3 | — | | |
| 11 | 22.0 | 1.43 | m | |
| | | 1.58 | m | |
| 12 | 40.7 | 1.46 | m | |
| | | 1.60 | m | |
| 13 | 48.3 | — | | |
| 14 | 85.3 | — | | |
| 15 | 33.0 | 1.72 | m | |
| | | 2.05 | m | |
| 16 | 28.7 | 1.82 | m | |
| | | 2.28 | m | |
| 17 | 51.4 | 2.57 | m | |
| 18 | 18.2 (18.5)** | 1.13 s (1.18 s)** | | (3H) |
| 19 | 16.7 | 0.84 | s | (3H) |
| 20 | 122.9 | — | | |
| 21 | 148.8 | 7.33 | d | 1.9 |
| 22 | 147.1 | 7.92 | dd | 9.4; 1.9 |
| 23 | 115.6 | 6.37 | d | 9.4 |
| 24 | 162.7 | — | | |
| 1′ | 73.6 | 4.12 | m | (2H) |
| 2′ | 29.0 | 1.78 | m | (2H) |
| 3′ | 24.1 | 1.48 | m | (2H) |
| 4′ | 24.4 | 1.82 | m | (2H) |
| 5′ | 57.9 | 2.81 | m | (2H) |
| 6′/10′ | 53.3 | 2.94 | m | (4H) |
| 7′/9′ | 23.7 | 1.93 | m | (4H) |
| 8′ | 23.2 | 1.64 | m | (2H) |

*Solvent: CDCl₃ (δ_{C} = 77.5 ppm; δ_{H} = 7.36 ppm)
**Signal of the minor stereoisomer

Compound 5

| Compound ID | BP249 |
|---|---|
| Name | 5-((10R,13R,14S,17R)-3-(2-(4-benzylpiperidin-1-yl)ethoxyimino)- |
| | 14-hydroxy-10,13-dimethyl-2,3,6,7,8,9,10,11,12,13,14,15,16,17- |
| | tetradecahydro-1H-cyclopenta[α]phenanthren-17-yl)-2H-pyran-2- |
| | one |
| Molecular formula | C₃₈H₅₀N₂O₄ |
| Molecular weight [g/mol] | 598.83 |
| Purity (LC/MS-ELSD) | 100% |
| Comment | 3:2-mixture of E/Z-Isomers |
| Structure | 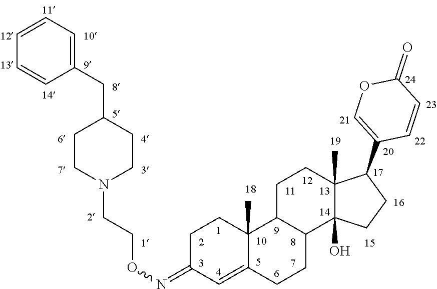 |

### LC/MS Assignment:

| | |
|---|---|
| (+)-ESI | (−)-ESI |

| m/z | Interpretation | m/z | Interpretation |
|---|---|---|---|
| 599 | [M + H]⁺ | 643 | [M + HCOO]⁻ |

### Assignment of the ¹H- and ¹³C-NMR-Signals* (Based on HH-COSY, HSQC, and HMBC Experiments)

| | | | |
|---|---|---|---|
| | | | J [Hz]/ |
| Position | ¹³C [ppm] | ¹H [ppm] | Int. |

| 1 | 35.3 | 1.45 | m | |
|---|---|---|---|---|
| | | 1.98 | m | |
| 2 | 19.9 | 2.17 | m | |
| | | 3.03 | m | |
| 3 | 156.0 | — | | |
| 4 | 118.0 (111.6)** | 5.88 s (6.46 s)** | | |
| 5 | 154.7 | — | | |
| 6 | 33.2 | 2.40 | m | (2H) |
| 7 | 28.1 | 1.26 | m | |
| | | 2.19 | m | |
| 8 | 43.1 | 1.66 | m | |
| 9 | 50.0 | 1.22 | m | |
| 10 | 38.3 | — | | |
| 11 | 22.0 | 1.45 | m | |
| | | 1.61 | m | |
| 12 | 40.9 | 1.48 | m | |
| | | 1.62 | m | |
| 13 | 48.5 | — | | |
| 14 | 85.4 | — | | |
| 15 | 33.2 | 1.71 | m | |
| | | 2.05 | m | |
| 16 | 28.9 | 1.84 | m | |
| | | 2.27 | m | |
| 17 | 51.3 | 2.57 | m | |
| 18 | 18.3 (18.5)** | 1.13 s (1.17 s)** | | (3H) |
| 19 | 16.7 | 0.84 | s | (3H) |
| 20 | 122.8 | — | | |
| 21 | 148.9 | 7.33 | d | 1.9 |
| 22 | 146.9 | 7.91 | dd | 9.4; 1.9 |
| 23 | 115.8 | 6.36 | d | 9.4 |
| 24 | 162.5 | — | | |
| 1′ | 71.5 | 4.36 | m | (2H) |
| 2′ | 57.5 | 2.91 | m | (2H) |
| 3′/7′ | 54.5 | 3.17 | m | (4H) |
| 4′/6′ | 31.7 | 1.77 | m | (4H) |
| 5′ | 37.8 | 1.67 | m | |
| 8′ | 43.4 | 2.65 | m | (2H) |
| 9′ | 140.7 | — | | |
| 10′/14′ | 129.5 | 7.24 | d | 7.3 (2H) |
| 11′/13′ | 128.5 | 7.37 | t | 7.3 (2H) |
| 12′ | 126.2 | 7.29 | t | 7.3 |

*Solvent: CDCl₃ (δ_{C} = 77.5 ppm; δ_{H} = 7.36 ppm)
**Signal of the minor stereoisomer

### Example 5

### Broad Spectrum Activity Against Human Cancer Cell Lines

By using the HIF-1α sensitive A549 sentinel line, the cell line may be incubated with the compound of the invention for 24 hours and reporter activity may be measured by FACS analysis.

### Example 6

### Inhibit Reporter Activity in A549 Sentinel Line

A dose response for each of the compound of the invention may be performed for each cell line and the IC₅₀ value determined.

### Example 7

### Inhibition of Induction of HIF-1α and HIF-2α During Hypoxia

Caki-1 (renal cancer), A549 (lung cancer), Panc-1 (pancreatic cancer) and Hep3B (liver cancer) cells may be treated with the compound of the invention under hypoxic conditions. The cells may be treated with each compound for 4 hours under normoxic (N, 20% O₂) or hypoxic (H, 1% O₂) conditions. Expression of HIF-1α, HIF-1β and β-actin and other proteins may be analyzed by Western blotting.

### Example 8

### Attenuation of Hypoxia Induced VEGF Secretion

Caki-1 cells may be treated with the compound of the invention and cultured under hypoxia for 16 hours. VEGF levels in conditioned medium may be measured using an ELISA kit.

### Example 9

### Inhibition Activity of Na-K-ATPase, the Physiological Receptor and the Pharmaceutical Target

Compounds of the invention may be tested for their activity on Na-K-ATPase enzyme in an in vitro enzyme assay. The ATPase activity may be assayed as the amount of inorganic phosphate liberated from ATP by Dog Kidney or Porcine cerebral cortex Na-K-ATPase.

### Example 10

### In Vitro Data for 3-Oximethers

In vitro data may be determined for the compounds of the invention in the following assays: APA (A549); APA (Caki-1); ATPase Inh Dog kidney; ATPase Inh Pig brain; AICAR-RA; AHA; and APA (Panc-1).

### Example 11

### Cell-based Na⁺, K⁺-ATPase CHO-K1 Screen

Compounds were screened using Ion Channel Research (ICR) technology and a non-radioactive Rubidium Uptake Assay

The test compounds were subjected to preparation of 100× stocks in 100% DMSO. The stocks were stored at 4° C. Digitoxin (Sigma Chemical Co.) was used as a positive control in the screens. No problem was encountered in the solubility of specific stock(s) or dose(s) in the test wells.

For all the test compounds and standard blockers, 100× solutions were used to prepare 1× test doses for Na⁺, K⁺-ATPase screen in CHO cell line. The final DMSO concentration in each well was 1%.

### Na+, K+-ATPase Screen Protocol:

1. Culture: The CHO-K1 cell line endogenously expressing Na⁺, K⁺-ATPase, was grown in Ham's F-12 supplemented with 10% FCS (Sigma), 100 μg/ml streptomycin/100,000 U/L penicillin (Sigma) at 37° C., in 5% CO₂. Cells were plated at a density of 50,000 cells/well in 96-well microplates and incubated at 37° C., 5% CO₂ until 80-90% confluency was attained (c. 24 hours).
2. Rb⁺ Uptake: Cells were washed with 200 μl of Rb⁺ Uptake Buffer once and R⁺ loaded by application of 198 μl R⁺ Uptake Buffer. Compounds were added at 2 μl/well of a 100× stock solution in 100% DMSO in the uptake buffer followed by incubation for 30 minutes at room temperature (c. 22° C.). The total volume in each well was 200 μl (198 μl of R⁺ Uptake Buffer and 2 μl of the 100× compound). The 100× compound solution gets diluted in R⁺ Uptake Buffer to 1× and the DMSO to 1%.
3. Wash: Residual R⁺ and compound were removed by four successive washes with 200 μl of SPA-Wash Buffer.
4. Cell Lysis: Intracellular samples were obtained by whole cell lysis with the application of 200 μl Lysis Solution.
5. Analysis: The level of R⁺ in the intracellular samples was measured by ICR8000 (Aurora Biomed Inc., Vancouver, Canada). using flame atomic absorption spectroscopy.

Reagents used were SPA-Wash Buffer: (5.4 mM KCl); SPA-Rb+ Uptake Buffer: (5.4 mM RbCl); Lysis Solution: (0.15% SDS).

Results:

The intracellular R⁺ content (mg/L) of each test dose of the test compounds was normalized to the detection window of inhibitory activity of digitoxin, used as a positive control, by calculating:

$$\frac{actual uptake - basal uptake}{window of detection} \times 100 \%$$

Further analysis of the normalized data was performed with Xlfit3 to draw the curve fits, and to obtain information on different parameters like Mean (n=3), SEM, inhibition (%), and IC₅₀ values of the test compounds. The Z′ value of the screen was calculated as 0.680.

Rank Order:

The compounds were ranked based on the potency (IC₅₀ value) against the screen target as presented in Table 2.

TABLE 2

| Test compound | IC₅₀ (μM) | Rank order |
|---|---|---|
| BP248 | 27.9 | 1 |
| BP247 | 47.4 | 2 |
| Digitoxin | 44.9 | 2 |
| BP249 | 41.9 | 2 |
| Proscillaridin A | 46.4 | 2 |
| BP246 | 57.8 | 3 |
| BP245 | 93.2 | 4 |

Inhibitory Profiles:

The inhibitory profile of each test compound follows.

### Compound 1: BP245 (IC₅₀=93.2 μM)

| | Dose Log[drug](M) | | | | | | | |
|---|---|---|---|---|---|---|---|---|
| | −6.3 | −6.0 | −5.3 | −5.0 | −4.3 | −4.0 | −3.7 | −3.3 |
| Dose (uM) | 0.50 | 1.0 | 5.0 | 10 | 50 | 100 | 200 | 500 |
| Normalized | 98.9 | 96.9 | 96.0 | 94.3 | 82.4 | 67.6 | 38.7 | 29.3 |
| Uptake (%) | | | | | | | | |
| Inhibition (%) | 0.0 | 2.9 | 4.2 | 6.6 | 23.7 | 44.9 | 86.5 | 100.0 |
| SEM | 1.3 | 0.7 | 2.3 | 2.3 | 2.6 | 1.4 | 1.7 | 1.6 |
| DMSO (%) | 1.0 | 1.0 | 1.0 | 1.0 | 1.0 | 1.0 | 1.0 | 1.0 |

| | |
|---|---|
| Sample | Good |
| Solubility | |

### Compound 2: BP246 (IC₅₀=57.8 μM)

| | Dose Log[drug](M) | | | | | | | |
|---|---|---|---|---|---|---|---|---|
| | −6.3 | −6.0 | −5.3 | −5.0 | −4.3 | −4.0 | −3.7 | −3.3 |
| Dose (uM) | 0.50 | 1.0 | 5.0 | 10 | 50 | 100 | 200 | 500 |
| Normalized | 95.3 | 93.4 | 84.8 | 76.6 | 58.7 | 39.0 | 17.5 | 8.2 |
| Uptake (%) | | | | | | | | |
| Inhibition (%) | 0.0 | 2.2 | 12.0 | 21.5 | 42.0 | 64.7 | 89.3 | 100.0 |
| SEM | 2.3 | 3.2 | 1.5 | 1.9 | 2.6 | 3.1 | 2.8 | 2.9 |
| DMSO (%) | 1.0 | 1.0 | 1.0 | 1.0 | 1.0 | 1.0 | 1.0 | 1.0 |

| | |
|---|---|
| Sample | Good |
| Solubility | |

### Compound 3: BP247 (IC₅₀=47.4 μM)

| | Dose Log[drug](M) | | | | | | | |
|---|---|---|---|---|---|---|---|---|
| | −6.3 | −6.0 | −5.3 | −5.0 | −4.3 | −4.0 | −3.7 | −3.3 |
| Dose (uM) | 0.50 | 1.0 | 5.0 | 10 | 50 | 100 | 200 | 500 |
| Normalized | 96.0 | 96.2 | 88.2 | 83.2 | 58.3 | 39.0 | 21.1 | 11.9 |
| Uptake (%) | | | | | | | | |
| Inhibition (%) | 0.0 | −0.3 | 9.2 | 15.2 | 44.9 | 67.7 | 89.0 | 100.0 |
| SEM | 1.8 | 1.1 | 1.0 | 1.6 | 0.6 | 1.6 | 1.6 | 1.5 |
| DMSO (%) | 1.0 | 1.0 | 1.0 | 1.0 | 1.0 | 1.0 | 1.0 | 1.0 |

| | |
|---|---|
| Sample | Good |
| Solubility | |

### Compound 4: BP248 (IC₅₀=27.9 μM)

| | Dose Log[drug](M) | | | | | | | |
|---|---|---|---|---|---|---|---|---|
| | −6.3 | −6.0 | −5.3 | −5.0 | −4.3 | −4.0 | −3.7 | −3.3 |
| Dose (uM) | 0.50 | 1.0 | 5.0 | 10 | 50 | 100 | 200 | 500 |
| Normalized | 104.5 | 105.6 | 96.6 | 81.1 | 51.5 | 31.6 | 23.1 | 17.9 |
| Uptake (%) | | | | | | | | |
| Inhibition (%) | 1.3 | 0.0 | 9.1 | 27.0 | 61.2 | 84.1 | 94.0 | 100.0 |
| SEM | 2.9 | 3.8 | 1.5 | 7.4 | 8.0 | 5.9 | 6.2 | 1.6 |
| DMSO (%) | 1.0 | 1.0 | 1.0 | 1.0 | 1.0 | 1.0 | 1.0 | 1.0 |

| | |
|---|---|
| Sample | Good |
| Solubility | |

### Compound 5: BP249 (IC₅₀=41.9 μM)

| | Dose Log[drug](M) | | | | | | | |
|---|---|---|---|---|---|---|---|---|
| | −6.3 | −6.0 | −5.3 | −5.0 | −4.3 | −4.0 | −3.7 | −3.3 |
| Dose (uM) | 0.50 | 1.0 | 5.0 | 10 | 50 | 100 | 200 | 500 |
| Normalized | 103.0 | 103.5 | 100.7 | 87.9 | 57.3 | 41.0 | 29.0 | 18.7 |
| Uptake (%) | | | | | | | | |
| Inhibition (%) | 0.6 | 0.0 | 2.6 | 17.9 | 54.2 | 73.4 | 87.8 | 100.0 |
| SEM | 2.3 | 2.1 | 1.5 | 2.6 | 2.2 | 6.4 | 1.2 | 1.3 |
| DMSO (%) | 1.0 | 1.0 | 1.0 | 1.0 | 1.0 | 1.0 | 1.0 | 1.0 |

| | |
|---|---|
| Sample | Good |
| Solubility | |

### Proscillaridin A (IC₅₀=46.4 μM)

| | Dose Log[drug](M) | | | | | | | |
|---|---|---|---|---|---|---|---|---|
| | −6.3 | −6.0 | −5.3 | −5.0 | −4.3 | −4.0 | −3.7 | −3.3 |
| Dose (uM) | 0.50 | 1.0 | 5.0 | 10 | 50 | 100 | 200 | 500 |
| Normalized | 99.4 | 100.3 | 93.8 | 80.5 | 54.6 | 44.0 | 38.2 | 28.7 |
| Uptake (%) | | | | | | | | |
| Inhibition (%) | 1.3 | 0.0 | 9.0 | 20.6 | 51.8 | 69.8 | 86.7 | 100.0 |
| SEM | 1.3 | 2.8 | 5.3 | 2.9 | 1.3 | 1.1 | 0.2 | 0.1 |
| DMSO (%) | 1.0 | 1.0 | 1.0 | 1.0 | 1.0 | 1.0 | 1.0 | 1.0 |

| | |
|---|---|
| Sample | Good |
| Solubility | |

### Digitoxin (IC₅₀=44.9 μM)

| | Dose Log[drug](M) | | | | | | | |
|---|---|---|---|---|---|---|---|---|
| | −6.3 | −6.0 | −5.3 | −5.0 | −4.3 | −4.0 | −3.3 | −3.0 |
| Dose (uM) | 0.50 | 1.0 | 5.0 | 10 | 50 | 100 | 500 | 1000 |
| Normalized | 100.0 | 93.8 | 91.4 | 80.2 | 48.1 | 23.2 | 10.3 | 0.0 |
| Uptake (%) | | | | | | | | |
| Inhibition (%) | 0.0 | 6.2 | 8.6 | 19.8 | 51.9 | 76.8 | 89.7 | 100.0 |
| SEM | 5.4 | 2.7 | 5.1 | 3.9 | 4.1 | 1.0 | 0.6 | 0.7 |
| DMSO (%) | 1.0 | 1.0 | 1.0 | 1.0 | 1.0 | 1.0 | 1.0 | 1.0 |

| | |
|---|---|
| Sample | Good |
| Solubility | |

### Example 12

### Cell-Based Na⁺, K⁺-ATPase HEK-293 Screen

The procedure was generally as in Example 11, with the exception of the culture used in the Na⁺, K⁺-ATPase screen protocol:

1. Culture: The HEK-293 cell line endogenously expressing Na⁺, K⁺-ATPase was grown in DMEM supplemented with 10% FCS (Sigma), 100 μg/ml Strep/100,000 U/L Pen (Sigma) at 37° C., in 5% CO₂. Cells were plated at a density of 50,000 cells/well in 96-well microplates and incubated at 37° C., 5% CO₂ until 80-90% confluency was attained

Results:

The Z′ value of the screen was calculated as 0.791.

Rank Order:

The compounds were ranked based on the potency (IC₅₀ value) against the screen target as presented in Table 3.

TABLE 3

| Test compound | IC₅₀ (μM) | Rank order |
|---|---|---|
| BP247 | 0.183 | 1 |
| Proscillaridin A | 0.240 | 1 |
| BP246 | 0.335 | 1 |
| BP245 | 0.732 | 2 |
| Digitoxin | 1.097 | 2 |
| BP249 | 5.535 | 4 |
| BP248 | 5.549 | 4 |

Inhibitory Profiles:

The inhibitory profile of each test compound follows.

### Compound 1: BP245 (IC₅₀=0.732 μM)

| | Dose Log[drug](M) | | | | | | | |
|---|---|---|---|---|---|---|---|---|
| | −8.0 | −7.3 | −7.0 | −6.3 | −6.0 | −5.3 | −5.0 | −4.3 |
| Dose (uM) | 0.01 | 0.1 | 0.1 | 1 | 1 | 5 | 10 | 50 |
| Normalized | 103.6 | 95.3 | 81.4 | 67.4 | 47.3 | 10.8 | 9.0 | 3.1 |
| Uptake (%) | | | | | | | | |
| Inhibition (%) | 0.0 | 8.3 | 22.1 | 36.0 | 56.0 | 92.3 | 94.1 | 100.0 |
| SEM | 2.7 | 2.0 | 5.0 | 4.9 | 5.4 | 3.8 | 2.5 | 1.3 |
| DMSO (%) | 1.0 | 1.0 | 1.0 | 1.0 | 1.0 | 1.0 | 1.0 | 1.0 |

| | |
|---|---|
| Sample | Good |
| Solubility | |

### Compound 2: BP246 (IC₅₀=0.335 μM)

| | Dose Log[drug](M) | | | | | | | |
|---|---|---|---|---|---|---|---|---|
| | −8.0 | −7.3 | −7.0 | −6.3 | −6.0 | −5.3 | −5.0 | −4.3 |
| Dose (uM) | 0.01 | 0.1 | 0.1 | 1 | 1 | 5 | 10 | 50 |
| Normalized | 98.7 | 84.6 | 69.8 | 49.5 | 18.5 | 1.0 | 1.2 | −2.7 |
| Uptake (%) | | | | | | | | |
| Inhibition (%) | 0.0 | 13.9 | 28.5 | 48.5 | 79.1 | 96.3 | 96.1 | 100.0 |
| SEM | 4.4 | 6.2 | 5.5 | 4.6 | 5.7 | 2.8 | 0.6 | 1.5 |
| DMSO (%) | 1.0 | 1.0 | 1.0 | 1.0 | 1.0 | 1.0 | 1.0 | 1.0 |

| | |
|---|---|
| Sample | Good |
| Solubility | |

### Compound 3: BP247 (IC₅₀=0.183 μM)

| | Dose Log[drug](M) | | | | | | | |
|---|---|---|---|---|---|---|---|---|
| | −8.0 | −7.3 | −7.0 | −6.3 | −6.0 | −5.3 | −5.0 | −4.3 |
| Dose (uM) | 0.01 | 0.1 | 0.1 | 1 | 1 | 5 | 10 | 50 |
| Normalized | 91.2 | 75.2 | 49.2 | 27.3 | 16.5 | 2.5 | 3.8 | −2.3 |
| Uptake (%) | | | | | | | | |
| Inhibition (%) | 0.0 | 17.1 | 44.9 | 68.4 | 79.9 | 94.8 | 93.5 | 100.0 |
| SEM | 8.5 | 6.6 | 8.1 | 8.7 | 1.3 | 3.8 | 4.1 | 2.8 |
| DMSO (%) | 1.0 | 1.0 | 1.0 | 1.0 | 1.0 | 1.0 | 1.0 | 1.0 |

| | |
|---|---|
| Sample | Good |
| Solubility | |

### Compound 4: BP248 (IC₅₀=5.549 μM)

| | Dose Log[drug](M) | | | | | | | |
|---|---|---|---|---|---|---|---|---|
| | −8.0 | −7.3 | −7.0 | −6.3 | −6.0 | −5.3 | −5.0 | −4.3 |
| Dose (uM) | 0.01 | 0.1 | 0.1 | 1 | 1 | 5 | 10 | 50 |
| Normalized | 56.6 | 56.1 | 55.4 | 53.5 | 49.6 | 25.5 | 5.0 | −13.1 |
| Uptake (%) | | | | | | | | |
| Inhibition (%) | 0.0 | 0.8 | 1.7 | 4.5 | 10.1 | 44.7 | 74.0 | 100.0 |
| SEM | 1.5 | 2.8 | 3.0 | 0.8 | 3.9 | 5.1 | 2.1 | 4.9 |
| DMSO (%) | 1.0 | 1.0 | 1.0 | 1.0 | 1.0 | 1.0 | 1.0 | 1.0 |

| | |
|---|---|
| Sample | Good |
| Solubility | |

### Compound 5: BP249 (IC₅₀=5.535 μM)

| | Dose Log[drug](M) | | | | | | | |
|---|---|---|---|---|---|---|---|---|
| | −8.0 | −7.3 | −7.0 | −6.3 | −6.0 | −5.3 | −5.0 | −4.3 |
| Dose (uM) | 0.01 | 0.1 | 0.1 | 1 | 1 | 5 | 10 | 50 |
| Normalized | 93.9 | 91.4 | 88.2 | 85.5 | 75.7 | 49.6 | 37.5 | 0.1 |
| Uptake (%) | | | | | | | | |
| Inhibition (%) | 0.0 | 2.7 | 6.1 | 9.0 | 19.4 | 47.2 | 60.1 | 100.0 |
| SEM | 1.1 | 3.7 | 1.7 | 4.3 | 4.6 | 2.9 | 4.0 | 2.4 |
| DMSO (%) | 1.0 | 1.0 | 1.0 | 1.0 | 1.0 | 1.0 | 1.0 | 1.0 |

| | |
|---|---|
| Sample | Good |
| Solubility | |

### Proscillaridin A (IC₅₀=0.240 μM)

| | Dose Log[drug](M) | | | | | | | |
|---|---|---|---|---|---|---|---|---|
| | −8.0 | −7.3 | −7.0 | −6.3 | −6.0 | −5.3 | −5.0 | −4.3 |
| Dose (uM) | 0.01 | 0.1 | 0.1 | 1 | 1 | 5 | 10 | 50 |
| Normalized | 100.0 | 82.3 | 58.9 | 40.7 | 24.2 | 8.4 | 4.8 | 0.0 |
| Uptake (%) | | | | | | | | |
| Inhibition (%) | 0.0 | 17.7 | 41.1 | 59.3 | 75.8 | 91.6 | 95.2 | 100.0 |
| SEM | 2.4 | 3.9 | 4.6 | 4.3 | 3.0 | 3.2 | 1.6 | 3.6 |
| DMSO (%) | 1.0 | 1.0 | 1.0 | 1.0 | 1.0 | 1.0 | 1.0 | 1.0 |

| | |
|---|---|
| Sample | Good |
| Solubility | |

### Digitoxin (IC₅₀=1.097 μM)

| | Dose Log[drug](M) | | | | | | | |
|---|---|---|---|---|---|---|---|---|
| | −7.3 | −7.0 | −6.3 | −6.0 | −5.3 | −5.0 | −4.3 | −4.0 |
| Dose (uM) | 0.05 | 0.1 | 0.5 | 1 | 5 | 10 | 50 | 100 |
| Normalized | 100.0 | 88.3 | 66.5 | 49.9 | 21.9 | 9.1 | 7.4 | 0.0 |
| Uptake (%) | | | | | | | | |
| Inhibition (%) | 0.0 | 11.7 | 33.5 | 50.1 | 78.1 | 90.9 | 92.6 | 100.0 |
| SEM | 2.3 | 3.9 | 2.3 | 2.5 | 5.7 | 3.1 | 4.6 | 3.9 |
| DMSO (%) | 1.0 | 1.0 | 1.0 | 1.0 | 1.0 | 1.0 | 1.0 | 1.0 |

| | |
|---|---|
| Sample | Good |
| Solubility | |

1. A compound of formula I: 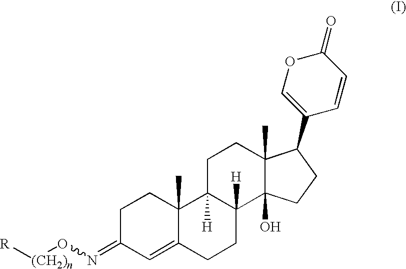
or a pharmaceutically acceptable salt or prodrug thereof, wherein:
R is a nitrogen-containing C₂₋₆ heterocyclyl group joined to the (CH₂)ₙ linkage by means of its nitrogen atom and optionally substituted with a C₁₋₅ alkyl, C₁₋₅ hydroxyalkyl, C₁₋₅ halogenoalkyl or C₁₋₅ acyl group, a phenyl group or a C₁₋₅ alkyl group further substituted with a phenyl group,
R is a group R′R″N— with R′ and R″ independently equal to H, C₁₋₅ alkyl or methylenecyclopropyl, and
n is an integer in the range 2 to 5,
with the proviso that, when R represents an unsubstituted piperidino group or a dimethylamino group, then n does not equal 2.

2. A compound as claimed in claim 1, wherein R is a group R¹R²N— with R¹ and R² independently equal to H, C₁₋₅ alkyl or methylenecyclopropyl, or R¹ and R² together with the nitrogen atom connecting them equal to: 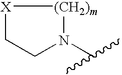
wherein X is CH₂, CH.CH₃, CH.Ph, CH.CH₂.Ph, O or NR³ where R³ is H, C₁₋₅ alkyl, C₁₋₅ hydroxyalkyl, C₁₋₅ halogenoalkyl or C₁₋₅ acyl, and m is 1, 2 or 3.

3. A compound as claimed in claim 1 or 2, wherein R is pyrrolidino, piperidino, morpholino, piperazino or homopiperazino, optionally substituted with a C₁₋₅ alkyl, C₁₋₅ hydroxyalkyl, C₁₋₅ halogenoalkyl or C₁₋₅ acyl group, a phenyl group or a C₁₋₅ alkyl group further substituted with a phenyl group.

4. A compound as claimed in claim 3, wherein the compound is of formula Ia: 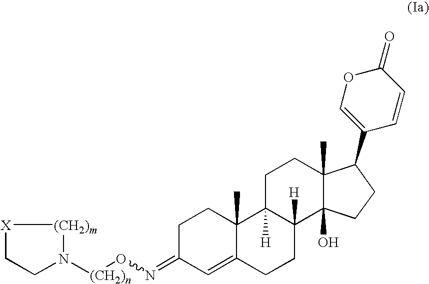
or a pharmaceutically acceptable salt or prodrug thereof, wherein X is CH₂, CH.CH₃, CH.Ph, CH.CH₂.Ph, O or NR³ where R³ is H, C₁₋₅ alkyl, C₁₋₅ hydroxyalkyl, C₁₋₅ halogenoalkyl or C₁₋₅ acyl, n is an integer in the range 2 to 5 and m is 1, 2 or 3.

5. A compound as claimed in claim 4, wherein m is 2 and X is CH₂, CH.CH₂.Ph, or O.

6. A compound as claimed in claim 4 or 5, wherein n is an integer in the range 3 to 5.

7. A method for treating a disorder in a mammal mediated by hypoxia inducible factor-1 (HIF-1), said method comprising administering to said mammal a compound of any one of claims 1 to 6, in an amount sufficient to treat said disorder.

8. A method for reducing VEGF expression in a cell, said method comprising contacting said cell with a compound of any of claims 1 to 6, in an amount sufficient to reduce said VEGF expression.

9. A method for treating a patient with a neoplastic disorder, said method comprising administering to said patient (i) a compound of a any one of claims 1 to 6, and (ii) an antiproliferative agent, wherein said compound, and said antiproliferative agent are administered simultaneously, or within 14 days of each other, each in an amount that together is sufficient to treat said neoplastic disorder.

10. A kit comprising:
(i) a compound of any one of claims 1 to 6; and
(ii) instructions for administering said compound to a patient diagnosed with a disorder mediated by hypoxia inducible factor-1 (HIF-1).

11. A method for synthesizing a compound of any one of claims 1 to 6, said method comprising the step of condensing H₂NO(CH₂)ₙR with the corresponding 3-oxo bufadienolide compound.
